SEQUENCE LISTING (32 sequences)

SEQ ID NO: 1 - DNA, 1023 bp, Saccharomyces cerevisiae
  CDS (1)..(1023)
atgaccgcaccattggtagtattgggtaacccacttttagatttccaagccgacgtcacggctgaatacctggccaagtattctctaaaggaaaacgacgcaattttggtcgatgccaaatcaggcgatgctaagatggctatttttgacgagctcttacagatgccagaaacaaagcttgttgcaggtggtgctgctcaaaacactgctagaggggcagcatacgttttgggcgccggccaggtggtgtacttcggttccgtcggtaaggacaagttcagcgagagattgcttaacgaaaacgaaaaagctggtgtcaagtctatgtaccaagttcaaaatgatattggtaccggtaagtgtgccgcattaatcactggccataaccggtccttggtcactgacttgggtgctgccaatttctttactccagaccacttggacaagcattgggacttggtcgaagcagctaagctcttctacatcggtggtttccacttgaccgtgtctccagacgctatcgttaagttgggccaacatgctaaagagaacagcaaacctttcgtgttgaactttagtgctcctttcattcctcatgtcttcaaagacgcattggccagagttttgccttatgctaccgtcatcatcgctaacgaatcggaggccgaagccttttgcgacgccttccaattagactgtgccaacactgatttggaagctattgctcaaagaattgtcaaggactctccagttgaaaagactgtcatcttcacccacggtgtcgaaccaacagtggtcgtgtcctccaagggtaccagcacatatccagtcaaacctttggactcttctaagatcgtcgacaccaacggtgctggtgacgccttcgctggtggctttatggctgggttgactaaaggtgaagatttggaaacctctattgacatgggtcaatggctagctgctttgtctattcaagaagttggtccctcttacccttccgaaaaaatatcttactctaaatag

SEQ ID NO: 2 - protein, 340 aa, Saccharomyces cerevisiae
MTAPLVVLGNPLLDFQADVTAEYLAKYSLKENDAILVDAKSGDAKMAIFDELLQMPETKLVAGGAAQNTARGAAYVLGAGQVVYFGSVGKDKFSERLLNENEKAGVKSMYQVQNDIGTGKCAALITGHNRSLVTDLGAANFFTPDHLDKHWDLVEAAKLFYIGGFHLTVSPDAIVKLGQHAKENSKPFVLNFSAPFIPHVFKDALARVLPYATVIIANESEAEAFCDAFQLDCANTDLEAIAQRIVKDSPVEKTVIFTHGVEPTVVVSSKGTSTYPVKPLDSSKIVDTNGAGDAFAGGFMAGLTKGEDLETSIDMGQWLAALSIQEVGPSYPSEKISYSK

SEQ ID NO: 3 - DNA, 993 bp, Saccharomyces cerevisiae
  CDS (1)..(993)
atggctagtaacgctgcaagggttgtcgctacagcgaaggattttgacaaagttgggttagggatcatcggatactatcttcagttatatgcggtggaattgatactgagtgaagaagatcggtcgcaagagatgaccgcactagcgacagaacttcttgatactattgaagcgtttaagaaggagattggcggcgagagtgaagcagaagacagtgacaaaagcttacacgttatgaacacgctaatacatgatcaagaaaaagccaaaatttatatgctaaattttacaatgtcgttgtataacgagaaactgaaacagctaaaagatggtccctgggatgttatgttaaaaagatcactttggtgttgtattgatttattttcatgcattttacacctgtggaaggaaaatataagtgaaacgagtacaaattcattacaaaaacgaattaagtattgcaaaatttatttgagtaaattggcaaagggtgaaattggttccagcgacgagaaaacgttagattatgcagattttgcggatgatagcgaggagattaaggatgaagatgttgatcaccagacaagcgatttggaaaacaataataatgataaagtcgaaggcctagccccaaaagaccaaacaacatcatacgagcctgtggatgaggtacctgaatttattgatgatgccgatagcgtaaacgaagaagaacagaccgtcgacaagaacgaagatgcaataacaaaagacgaacaacaagttgtgaagaaagaagtggatcttacgcgcccttctgcaccatcagaacctgctgcagcagagcataaatcgtatacaaaggatgagttgacaaaaattatggacagggccagtaaaatagagcaaattcaaaaacttgccaaatacgccataagcgcattgaattatgaagacctgcccaccgccaaagatgagttaactaaggcgttggatctgttaaattctatttga

SEQ ID NO: 4 - protein, 330 aa, Saccharomyces cerevisiae
MASNAARVVATAKDFDKVGLGIIGYYLQLYAVELILSEEDRSQEMTALATELLDTIEAFKKEIGGESEAEDSDKSLHVMNTLIHDQEKAKIYMLNFTMSLYNEKLKQLKDGPWDVMLKRSLWCCIDLFSCILHLWKENISETSTNSLQKRIKYCKIYLSKLAKGEIGSSDEKTLDYADFADDSEEIKDEDVDHQTSDLENNNNDKVEGLAPKDQTTSYEPVDEVPEFIDDADSVNEEEQTVDKNEDAITKDEQQVVKKEVDLTRPSAPSEPAAAEHKSYTKDELTKIMDRASKIEQIQKLAKYAISALNYEDLPTAKDELTKALDLLNSI

SEQ ID NO: 5 - DNA, 1155 bp, Saccharomyces cerevisiae
  CDS (1)..(1155)
atgtccaagagcaaaactttcttatttacctctgaatccgtcggtgaaggtcacccagacaagatttgtgaccaagtttctgatgctattttggacgcttgtttagaacaagatccattctccaaggttgcctgtgaaacagctgccaaaactggtatgattatggttttcggtgaaattaccaccaaagctagacttgactaccaacaaatagtaagagataccatcaagaagattggttatgacgattctgccaagggtttcgactacaagacatgtaatgttttagtagctatcgaacaacaatctccagatatcgctcaaggtctgcactatgaaaagagcttagaagacttaggtgctggtgaccaaggtataatgtttggttacgctacagacgaaactccagaagggttaccattgaccattcttttggctcacaaattgaacatggctatggcagatgctagaagagatggttctctcccatggttgagaccagacacaaagactcaagtcactgtcgaatacgaagacgacaatggtagatgggttccaaagaggatagataccgttgttatttctgctcaacatgctgatgaaatttccaccgctgacttgagaactcaacttcaaaaagatattgttgaaaaggtcataccaaaggatatgttagacgaaaataccaaatatttcatccaaccatccggtagattcgtcatcggtggtcctcaaggtgacgctggtttgaccggtagaaagattattgtcgacgcttacggtggtgcctcatccgtcggtggtggtgccttctccggtaaggactattccaaggtcgatcgttccgctgcttacgctgctagatgggttgccaagtctctagttgccgctggtttgtgtaagagagtccaagtccaattttcatatgctattggtattgctgaaccattgtctttacatgtggacacctatggtacagctacaaaatcagatgacgaaatcattgaaattattaagaagaacttcgacttgagaccaggtgtgttagtaaaggaattagatttggctagaccaatttacttaccaaccgcttcttatggtcacttcactaatcaagagtactcatgggaaaaaccaaagaaattggaattttaa

SEQ ID NO: 6 - protein, 384 aa, Saccharomyces cerevisiae
MSKSKTFLFTSESVGEGHPDKICDQVSDAILDACLEQDPFSKVACETAAKTGMIMVFGEITTKARLDYQQIVRDTIKKIGYDDSAKGFDYKTCNVLVAIEQQSPDIAQGLHYEKSLEDLGAGDQGIMFGYATDETPEGLPLTILLAHKLNMAMADARRDGSLPWLRPDTKTQVTVEYEDDNGRWVPKRIDTVVISAQHADEISTADLRTQLQKDIVEKVIPKDMLDENTKYFIQPSGRFVIGGPQGDAGLTGRKIIVDAYGGASSVGGGAFSGKDYSKVDRSAAYAARWVAKSLVAAGLCKRVQVQFSYAIGIAEPLSLHVDTYGTATKSDDEIIEIIKKNFDLRPGVLVKELDLARPIYLPTASYGHFTNQEYSWEKPKKLEF

SEQ ID NO: 7 - DNA, 1764 bp, Saccharomyces cerevisiae
  CDS (1)..(1764)
atggatatactcaagaggggaaatgaatcggacaagtttacgaaaatagagacagaatctactacgataccaaatgactcggatagatctggttcactgatcagaagaatgaaggattccttcaagcaaagtaacctgcatgtcattccagaagaccttgaaaacagcgaacagacagagcaggaaaaaatccaatggaaactagcttctcagccctatcaaaaagtcttgagccaaaggcacttgaccatgattgccataggtggtactttggggacgggactgttcattggtttaggttattctttggcatctgggccagccgctttgctaatcggttttttgttagttggtacttcaatgttctgtgtcgttcagagtgccgcagagctttcctgccaattcccggtttctggctcatatgccacacatgtcagtaggttcatagatgaatcggttggctttactgtggctacaaactatgctttggcttggctgatttcttttccaagcgaattaattggttgtgcacttactatttcatactggaaccaaacggttaatccggctgtttgggtggccattttttatgttttcattatggtactgaacttgtttggtgtgagaggctttgccgagactgagtttgctctgtcaatcattaaggttattgctatatttattttcatcattattggtatcgtccttattgccggaggggggcctaactctactggttatattggcgccaaatactggcacgacccaggtgccttcgcaaaacctgtcttcaagaatttgtgtaacacattcgtttctgctgctttttcctttggtggtagtgagttagtgctgttaactagtacagaatctaaaaatatttcagctatatcacgtgctgccaaaggtacgttctggagaatcgcgattttttacattactaccgttgtcattattggatgtctcgtaccttataacgaccccagactgctcagtggttcgaacagtgaggatgtatctgcctctccctttgttattgctttgagtaatacaggatcaatgggtgcaaaagtttccaactttatgaatgtcgttatccttgttgcggttgtgtcagtttgcaattcttgcgtttatgcttcttcaagactaattcaagctttaggtgcatctggccaacttccttcggtatgttcctacatggacagaaagggtaggcctttggttggcattgggattagtggtgcatttggtcttttaggttttcttgtggcctcgaaaaaggaggacgaggtcttcacttggcttttcgccctttgttccatttcatcattcttcacctggttctgtatttgtatgtcacaaattagatttaggatggctttgaaagctcaaggaagatccaatgacgaaatagcttacaaatccatactgggtgtttatggtggaattttggggtgtgtgctaaatgcattgctaattgcgggtgaaatatacgtatcggccgccccggtcggtagccctagctccgctgaagccttctttgaatactgtttaagtattccaatcatgattgttgtttattttgcacataggttttatcgaagagattggaaacacttctacatcaagaggagtgagatcgatctggatactgggtgttccgtggagaatctagagcttttcaaagcacaaaaagaagctgaggaacaactcattgcttctaagccattctattacaaaatctacagattttggtgttag

SEQ ID NO: 8 - protein, 587 aa, Saccharomyces cerevisiae
MDILKRGNESDKFTKIETESTTIPNDSDRSGSLIRRMKDSFKQSNLHVIPEDLENSEQTEQEKIQWKLASQPYQKVLSQRHLTMIAIGGTLGTGLFIGLGYSLASGPAALLIGFLLVGTSMFCVVQSAAELSCQFPVSGSYATHVSRFIDESVGFTVATNYALAWLISFPSELIGCALTISYWNQTVNPAVWVAIFYVFIMVLNLFGVRGFAETEFALSIIKVIAIFIFIIIGIVLIAGGGPNSTGYIGAKYWHDPGAFAKPVFKNLCNTFVSAAFSFGGSELVLLTSTESKNISAISRAAKGTFWRIAIFYITTVVIIGCLVPYNDPRLLSGSNSEDVSASPFVIALSNTGSMGAKVSNFMNVVILVAVVSVCNSCVYASSRLIQALGASGQLPSVCSYMDRKGRPLVGIGISGAFGLLGFLVASKKEDEVFTWLFALCSISSFFTWFCICMSQIRFRMALKAQGRSNDEIAYKSILGVYGGILGCVLNALLIAGEIYVSAAPVGSPSSAEAFFEYCLSIPIMIVVYFAHRFYRRDWKHFYIKRSEIDLDTGCSVENLELFKAQKEAEEQLIASKPFYYKIYRFWC

SEQ ID NO: 9 - DNA, 978 bp, Saccharomyces cerevisiae
  CDS (1)..(978)
atggcacgtcttcctctaaagcagttcttagcggataaccccaaaaaagttcttgttcttgacggtggtcaaggaacagaactggaaaacagaggtatcaaagttgcaaatcccgtgtggtctactattccatttattagcgaatcattttggtctgatgagtcatctgctaacagaaaaattgtcaaagaaatgttcaacgatttcttgaatgctggcgcagaaatattgatgactacaacataccaaacgagttataaatcagtttctgaaaacaccccaatcagaactttatccgagtacaataaccttttaaacaggattgtcgatttttctcgtaattgtattggcgaagacaaatatttgattggctgtattggcccatggggtgctcatatttgtcgtgagtttacaggcgactatggtgctgagccagaaaatattgatttctaccaatacttcaagcctcagttggagaatttcaataaaaatgacaaattggatttgattgggtttgaaaccattcctaacatccatgaactgaaagctatcttatcttgggatgagagtatcctgtctagacccttctatatcgggttgtctgtgcatgagcacggtgtcttgagagacggcactaccatggaagaaatcgcacaagttattaaggacttgggcgacaaaataaatcctaacttctcgttcttaggaatcaactgcgtcagcttcaaccaatcacccgacattcttgagtctctacatcaagcactaccaaatatggccttgcttgcttatccaaacagtggtgaagtttatgatactgaaaagaagatatggttgccaaatagcgataagctgaacagttgggatacggttgttaaacagtacattagcagcggtgcccgtatcattggtggttgttgcagaacaagtccaaaagacatccaagagatttctgcagccgtcaagaaatacacgtaa

SEQ ID NO: 10 - protein, 325 aa, Saccharomyces cerevisiae
MARLPLKQFLADNPKKVLVLDGGQGTELENRGIKVANPVWSTIPFISESFWSDESSANRKIVKEMFNDFLNAGAEILMTTTYQTSYKSVSENTPIRTLSEYNNLLNRIVDFSRNCIGEDKYLIGCIGPWGAHICREFTGDYGAEPENIDFYQYFKPQLENFNKNDKLDLIGFETIPNIHELKAILSWDESILSRPFYIGLSVHEHGVLRDGTTMEEIAQVIKDLGDKINPNFSFLGINCVSFNQSPDILESLHQALPNMALLAYPNSGEVYDTEKKIWLPNSDKLNSWDTVVKQYISSGARIIGGCCRTSPKDIQEISAAVKKYT

SEQ ID NO: 11 - DNA, 975 bp, Saccharomyces cerevisiae
  CDS (1)..(975)
atgaagcgcattccaatcaaagaactaatagttgagcaccccggaaaagttcttatccttgatggtggacagggtacagaattggaaaacagaggcattaacataaatagtccggtatggtctgcagctccttttacgagcgaatccttttgggagccatcttctcaagagcgaaaggtggtagaagaaatgtacagagactttatgattgctggcgcaaacatattaatgacaattacttaccaggcaaactttcaaagcatatctgagaatacctcgattaaaactctggctgcttacaagcgttttctcgataaaatcgtgtcatttactcgtgaatttattggtgaggaaaggtacttaatcgggagtattggcccatgggcagcacatgtatcctgtgaatatactggtgactatggtccccatcctgagaatattgattactacggctttttcaaaccccagctggagaacttcaaccaaaatagagatattgatcttattggttttgaaacgattccaaattttcatgagttaaaggctattttatcctgggatgaagatattatttcgaagcccttttatattgggttgtcggtggatgacaatagtttgctacgagacggtaccactttggaagaaatttctgtccatataaaaggcctcggaaataaaattaacaagaatctcttattaatgggagttaactgtgtcagtttcaatcaatcggcattaattcttaaaatgttgcacgagcatctacctggcatgcctctgctagtttacccaaacagtggagaaatctacaatcccaaagagaagacatggcaccggccgactaataagttggatgactgggagaccacggttaagaaattcgttgataatggtgcgcgcattattggcggttgttgtagaacgtctcctaaagatatcgccgaaattgcatcagctgtagataaatactcctaa

SEQ ID NO: 12 - protein, 324 aa, Saccharomyces cerevisiae
MKRIPIKELIVEHPGKVLILDGGQGTELENRGININSPVWSAAPFTSESFWEPSSQERKVVEEMYRDFMIAGANILMTITYQANFQSISENTSIKTLAAYKRFLDKIVSFTREFIGEERYLIGSIGPWAAHVSCEYTGDYGPHPENIDYYGFFKPQLENFNQNRDIDLIGFETIPNFHELKAILSWDEDIISKPFYIGLSVDDNSLLRDGTTLEEISVHIKGLGNKINKNLLLMGVNCVSFNQSALILKMLHEHLPGMPLLVYPNSGEIYNPKEKTWHRPTNKLDDWETTVKKFVDNGARIIGGCCRTSPKDIAEIASAVDKYS

SEQ ID NO: 13 - DNA, 1788 bp, Saccharomyces cerevisiae
  CDS (1)..(1788)
atgtcgtcgtcgaagtctctatacgaactgaaagacttgaaaaatagctccacagaaatacatgccacggggcaggataatgaaattgaatatttcgaaacaggctccaatgaccgtccatcctcacaacctcatttaggttacgaacagcataacacttctgccgtgcgtaggtttttcgactcctttaaaagagcggatcagggtccacaggatgaagtagaagcaacacaaatgaacgatcttacgtcggctatctcaccttcttctagacaggctcaagaactagaaaaaaatgaaagttcggacaacataggcgctaatacaggtcataagtcggactcgctgaagaaaaccattcagcctagacatgttctgatgattgcgttgggtacgggtatcggtactgggttattggtcggtaacggtaccgcgttggttcatgcgggtccagctggactacttattggttacgctattatgggttctatcttgtactgtattattcaagcatgtggtgaaatggcgctagtgtatagtaacttgactggtggctacaatgcataccccagtttccttgtggatgatggttttgggtttgcagtcgcttgggtttattgtttgcaatggctgtgtgtgtgtcctctggaattggtgaccgcatccatgactatcaaatattggacgacatctgtgaacccggatgtgttcgtcattattttctatgttttggtgattactattaatattttcggtgctcgtggttatgcagaagctgagttcttcttcaactgttgcaaaattttgatgatgactgggttcttcattcttggtattatcatcgatgttggtggcgctggtaatgatggttttattggtggtaaatactggcacgatccgggcgctttcaatggtaaacatgccattgacagatttaaaggtgttgctgcaacattagtgactgctgcttttgcctttggtggttcagagtttattgccatcaccactgcagaacaatctaatccaagaaaggccattccaggtgcggccaaacaaatgatctacagaatcttattcctattcttggctaccattattctactgggtttcttggtgccatacaattccgatcaattattgggttctaccggtggtggtactaaagcctcgccatatgtcattgctgttgcatcccacggtgtccgtgtcgtcccacacttcattaacgccgttattctactttccgtgctgtccatggctaactcctccttctactccagtgctcgtttatttttaactctatccgagcaaggttacgctcctaaggttttctcctacatcgacagagccggtagaccattgattgccatgggtgtttctgcattgtttgccgttattgccttctgtgctgcatctcccaaggaagaacaagttttcacttggttattggccatttctggtttgtctcagcttttcacatggactgccatttgtttatcccatcttagatttagaagagccatgaaagtccaagggagatctcttggagaattgggtttcaaatctcaaactggtgtttggggatctgcctacgcttgcattatgatgattttaattcttattgcccaattttgggtcgctatcgcccccattggtgaaggtaagctggatgcacaagcctttttcgaaaactacttggctatgccaatcttgattgcactttatgtcggctacaaggtctggcacaaggattggaaactgttcatcagggagacaagatcgacctag

SEQ ID NO: 14 - protein, 595 aa, Saccharomyces cerevisiae
MSSSKSLYELKDLKNSSTEIHATGQDNEIEYFETGSNDRPSSQPHLGYEQHNTSAVRRFFDSFKRADQGPQDEVEATQMNDLTSAISPSSRQAQELEKNESSDNIGANTGHKSDSLKKTIQPRHVLMIALGTGIGTGLLVGNGTALVHAGPAGLLIGYAIMGSILYCIIQACGEMALVYSNLTGGYNAYPSFLVDDGFGFAVAWVYCLQWLCVCPLELVTASMTIKYWTTSVNPDVFVIIFYVLVITINIFGARGYAEAEFFFNCCKILMMTGFFILGIIIDVGGAGNDGFIGGKYWHDPGAFNGKHAIDRFKGVAATLVTAAFAFGGSEFIAITTAEQSNPRKAIPGAAKQMIYRILFLFLATIILLGFLVPYNSDQLLGSTGGGTKASPYVIAVASHGVRVVPHFINAVILLSVLSMANSSFYSSARLFLTLSEQGYAPKVFSYIDRAGRPLIAMGVSALFAVIAFCAASPKEEQVFTWLLAISGLSQLFTWTAICLSHLRFRRAMKVQGRSLGELGFKSQTGVWGSAYACIMMILILIAQFWVAIAPIGEGKLDAQAFFENYLAMPILIALYVGYKVWHKDWKLFIRETRST

SEQ ID NO: 15 - DNA, 1830 bp, Saccharomyces cerevisiae
  CDS (1)..(1830)
atgctatcttcagaagattttggatcttctgggaaaaaggaaacttctcctgattcgatatcgatacgttcctttagtgccgggaataatttccaatcatcatcaagtgagaaaacttattctaagcaaaaatccgggagtgacaaacttatacatagatttgcggattcattcaaaagagccgagggtagcactacaagaactaagcaaataaatgaaaatacgtctgatttagaggatggcgttgagtctatcacgtcggattccaagttgaaaaagtccatgaagtcgcgccatgttgtcatgatgtctttagggacaggtattgggactggtcttttggtagctaatgcaaaaggtctacattacggtggtcctgctgcgctaataattggttacatcttggtttctttcgagacgtacttcatgatccaagctgcaggtgagatggcggtaacctatccgactttaccagcaaatttcaacgcatactcctccatattcatttccaaatcatttggattcgccacagtatggctttactgtttccaatggctaacggttttgcctttagagttaataaccgcgtctatgactattcaatttgggaatgataaaataaatccggacatttatattcttattttctatgttttcttagtattcattcatttcttcggtgtaaaagcctatggtgaaacggaattcatcttcaattgctgtaaaattttaatgattgcaggtttcattattctttctattgttatcaactgtggtggggccggaaatgacggttatatcggggccacttattggcataatccaggtgcttttgcaggtgacacatcgattggtaggttcaaaaacgtttgctatattttagttactgcttacttctcctttggtggtatggaattatttgcactaagtgttcaggaacagtctaaccctagaaaatctactccggtggcagccaagagaagcatttatcgtatcgttgtgatttatcttttgactatgatcctcattggattcaatgttccatataatgatgaccaactaatgggcgcaggcggatccgctacacatgcatctccctatgtcttagccgcttctattcacggtgtgaaaattgttccacatattatcaacgctgttattttgatttctgtggtttcagtggcaaattcctctttgtatgctggtccaagactgatttgctctttggcccaacaaggctacgcacccaagtttttagattacgttgacagagagggcaggcccttgagagctcttattgtgtgttgcgttttcggcgtcattgcttttgttgcagcttcatcaaaggaagagatcgtgtttacatggttagcagctatcgcaggcttgagtgaattattcacatggacttccataatgttgtcccatctgcgattcagacaagcaatgaaagtacagggaaggtctctagacgagttgggatacaaggccacaacagggatttggggttccatatacggtgtcttttttaatattttagtctttgttgcccaattttgggtagcattggcccccttaggtaatgggggcaaatgcgatgcggaatccttctttcaaaattatttagcttttccaatatggttggccttttacttcggatatatggtttacaaccgagattttacgctattaaatcccctcgacaagattgaccttgacttccacagacgcatttatgatccagagctaatgagacaagaggacgaagaaaataaagaaaaactaaggaatatgtctttgatgagaaaagcttatcatttctggtgttaa

SEQ ID NO: 16 - protein, 609 aa, Saccharomyces cerevisiae
MLSSEDFGSSGKKETSPDSISIRSFSAGNNFQSSSSEKTYSKQKSGSDKLIHRFADSFKRAEGSTTRTKQINENTSDLEDGVESITSDSKLKKSMKSRHVVMMSLGTGIGTGLLVANAKGLHYGGPAALIIGYILVSFETYFMIQAAGEMAVTYPTLPANFNAYSSIFISKSFGFATVWLYCFQWLTVLPLELITASMTIQFGNDKINPDIYILIFYVFLVFIHFFGVKAYGETEFIFNCCKILMIAGFIILSIVINCGGAGNDGYIGATYWHNPGAFAGDTSIGRFKNVCYILVTAYFSFGGMELFALSVQEQSNPRKSTPVAAKRSIYRIVVIYLLTMILIGFNVPYNDDQLMGAGGSATHASPYVLAASIHGVKIVPHIINAVILISVVSVANSSLYAGPRLICSLAQQGYAPKFLDYVDREGRPLRALIVCCVFGVIAFVAASSKEEIVFTWLAAIAGLSELFTWTSIMLSHLRFRQAMKVQGRSLDELGYKATTGIWGSIYGVFFNILVFVAQFWVALAPLGNGGKCDAESFFQNYLAFPIWLAFYFGYMVYNRDFTLLNPLDKIDLDFHRRIYDPELMRQEDEENKEKLRNMSLMRKAYHFWC

SEQ ID NO: 17 - DNA, 1815 bp, Saccharomyces cerevisiae
  CDS (1)..(1815)
atgtcagatcctatagtaacgtcttccaaaatggaaaaaagtgcagagtttgaagtaacagactctgctttatataataacttcaatacatcaacaacagcttcactaactccggagattaaggaacattctgaggaatctcgcaatgggttagttcacagattcgtcgactcattcagaagagccgaaagccaacgtttagaagaagacaatgacttggaggatggtaccaaatcgatgaaatctaataaccacttaaaaaagtcaatgaaatctagacatgttgtaatgatgtctttaggtacagggataggaacaggtcttctagttgccaacgccaaaggtttgagtcttgcaggcccggggtctttagtcatcggttacgttatggtgtccttcgtcacgtattttatggtccaagcggctggtgagatgggtgtcacttatccgacgcttccaggtaacttcaatgcatacaattcaatctttatttcaaagtcgtttgggttcgcgaccacttggttattttgcattcagtggttaactgtgctaccgttggaattgattacttcttccatgaccgtcaagtactggaatgacacaattaatgctgatgtattcattgtcattttctatgtgtttttattgtttatccacttttttggtgttaaggcatacggtgaaacggaatttatcttcaattcctgtaagatattgatggttgcaggattcattattttatcagtcgtcatcaattgtgggggagccggtgtggacggctatattggcggcaaatattggcgcgaccctggttcatttgcagaaggcagcggtgccactcgtttcaaagggatatgttatattctagtatctgcatatttctccttcggtggtattgaattatttgttctttccatcaatgagcagtctaaccctagaaaatccactcctgtagccgcgaagagaagtgtatatcgtattttgatcatttatttgctgacaatgattctaattgggttcaatgttcctcacaataacgatcaattgatgggttccggtggctccgcaacgcacgcttcaccatatgtcttggcagcctctattcacaaggttagggtcatcccccatatcattaatgctgttattttgatatcagtcatctctgttgcgaactctgccctttatgccgcaccgagattaatgtgttccttggctcaacagggctatgctccaaaatttttgaactatattgatagggagggaagacctttaagggccttggtagtgtgctctcttgttggtgtcgttggcttcgtcgcctgttctccacaggaagagcaagcatttacatggttggcagccattgcaggtttgagtgaacttttcacatggtcaggcattatgctttcacacattcgattcagaaaagcgatgaaagtccaaggaagatcacttgatgaagtcggctataaggccaataccggtatatggggttcatactatggggtttttttcaatatgcttgtctttatggcccaattttgggtcgcactgtctccaataggaaatggcggaaagtgtgatgctcaggcattcttcgagagttatctggctgccccgctatggatattcatgtatgttggatacatggtttacaaaagagattttacctttttaaacccactggacaagatcgacttggactttcatagaagagtttacgaccctgaaataatgaggcaggaggacgaagaaaataaagaaaggttaaagaactcttcaatattcgtcagagtctacaaattttggtgttag

SEQ ID NO: 18 - protein, 604 aa, Saccharomyces cerevisiae
MSDPIVTSSKMEKSAEFEVTDSALYNNFNTSTTASLTPEIKEHSEESRNGLVHRFVDSFRRAESQRLEEDNDLEDGTKSMKSNNHLKKSMKSRHVVMMSLGTGIGTGLLVANAKGLSLAGPGSLVIGYVMVSFVTYFMVQAAGEMGVTYPTLPGNFNAYNSIFISKSFGFATTWLFCIQWLTVLPLELITSSMTVKYWNDTINADVFIVIFYVFLLFIHFFGVKAYGETEFIFNSCKILMVAGFIILSVVINCGGAGVDGYIGGKYWRDPGSFAEGSGATRFKGICYILVSAYFSFGGIELFVLSINEQSNPRKSTPVAAKRSVYRILIIYLLTMILIGFNVPHNNDQLMGSGGSATHASPYVLAASIHKVRVIPHIINAVILISVISVANSALYAAPRLMCSLAQQGYAPKFLNYIDREGRPLRALVVCSLVGVVGFVACSPQEEQAFTWLAAIAGLSELFTWSGIMLSHIRFRKAMKVQGRSLDEVGYKANTGIWGSYYGVFFNMLVFMAQFWVALSPIGNGGKCDAQAFFESYLAAPLWIFMYVGYMVYKRDFTFLNPLDKIDLDFHRRVYDPEIMRQEDEENKERLKNSSIFVRVYKFWC

SEQ ID NO: 19 - DNA, 2019 bp, Saccharomyces cerevisiae
  CDS (1)..(2019)
atgaagcaggagcagtcccacgaaggcgactcatacagcacggaattcataaatctctttggcaaagataccgcaacacaccctagcagcaacaacggtgctaataataatggcatggggagcacgaactcgttggaccagtttgtggcaacagcctcatcgtcatcttctctggtgaccagcagcgagaataggcgccccctaataggtgacgttaccaataggggcaacactaacctatatgaccacgctgtcacgccagaaatactcttagaacagttggcctacgtggataacttcataccatctctggataacgagttctctaatgtggattggaatgtgaataccacccataataatgcaaacaataatggcgcggacactttcagcagcataaatgcaaatccttttgacttggatgaacaactagccattgagttgagtgcgtttgccgacgattctttcatcttcccagacgaggataagccgagcaataacaataacaacagcaataatggtaatgacgaccatagcaaccacgacgtattgcatgaggacccttctaccaataatagacaaagaaatcctcacttcttgactcaaagaaggaatactttcctaacttcccaatacgaccaatcaaagtctcgattttcgtccaaaaacaaaagaaatggcaataacggcgaaacaaacaactttggcgacaatatgcaaaataaccatccttttgagccaaactttatgggaagcccttctcagtttcccgctgacgcaactaatatgacatcaatcgaccatggcggcttcacaaatgttgacattacatcaactgagaacaatactaccggtgacaatggagtggatgcgctatcaaatctactacataggacaacacacacaccgaaccgctcctcccccctaagcaatgtcacttctgctcaaaattcctcttcacaacaacgaaaacattcggaaagcaaagtcgatagtaacagcgataataacagctccaacaaagcccccaatataactgttcctgactattcaattataccaacctctgttttagtaactctattaccgagggtcaacgtgcccaacggcgcatataactcgttgatcagcgcgggatttgacaatgatcaaatagatgctatagccgcaataatggcgtatcatcatcaaaaaaagattagggaaaataacagtaataataataaaaacatcaacaccaatgacagtcaagaggcacccattctaaaaaacatcaacgaacttttaagtgtcttaataccaccctctccggctgaaactcgtgggccaactaccttatcaacgtcgccttcgttcaatgagcacggtgtagtagcagaggcttcttttctaagctccattttggaactgggcataaagcatccaaaaagtaataatattcacaatcaacgacaaccttcacgaaacgatcataaaatatcaagagagagtgacggtaacaatggaaacgataatgtccatcataataacgctgttattaagtcaagtacgacgcgtggagacgaaattgccaagatacgatccgagccaactttaaatgcaagttcttctgatcacaaggaaaatagtttaaaaagatcacactccggagatttgaaaaataaaaaagtacccgtcgaccgcaagtattctgataatgaagacgatgaatatgacgatgcagatttacacggctttgaaaagaagcaactgatcaagaaagagttaggggacgacgatgaagatttattgatacagtcgaaaaaatctcatcaaaaaaaaaaactaaaggaaaaggagttagaatcatcgatacatgaactgaccgaaattgcagcatccttacaaaaacggatacatacgttagaaacggaaaacaagcttttaaagaatttagttctgagtagcggtgaaacggaaggaataaaaaaagctgaaagcttaaagaagcaaatttttgagaaggttcagaaagaataa

SEQ ID NO: 20 - protein, 672 aa, Saccharomyces cerevisiae
MKQEQSHEGDSYSTEFINLFGKDTATHPSSNNGANNNGMGSTNSLDQFVATASSSSSLVTSSENRRPLIGDVTNRGNTNLYDHAVTPEILLEQLAYVDNFIPSLDNEFSNVDWNVNTTHNNANNNGADTFSSINANPFDLDEQLAIELSAFADDSFIFPDEDKPSNNNNNSNNGNDDHSNHDVLHEDPSTNNRQRNPHFLTQRRNTFLTSQYDQSKSRFSSKNKRNGNNGETNNFGDNMQNNHPFEPNFMGSPSQFPADATNMTSIDHGGFTNVDITSTENNTTGDNGVDALSNLLHRTTHTPNRSSPLSNVTSAQNSSSQQRKHSESKVDSNSDNNSSNKAPNITVPDYSIIPTSVLVTLLPRVNVPNGAYNSLISAGFDNDQIDAIAAIMAYHHQKKIRENNSNNNKNINTNDSQEAPILKNINELLSVLIPPSPAETRGPTTLSTSPSFNEHGVVAEASFLSSILELGIKHPKSNNIHNQRQPSRNDHKISRESDGNNGNDNVHHNNAVIKSSTTRGDEIAKIRSEPTLNASSSDHKENSLKRSHSGDLKNKKVPVDRKYSDNEDDEYDDADLHGFEKKQLIKKELGDDDEDLLIQSKKSHQKKKLKEKELESSIHELTEIAASLQKRIHTLETENKLLKNLVLSSGETEGIKKAESLKKQIFEKVQKE

SEQ ID NO: 21 - DNA, 576 bp, Saccharomyces cerevisiae
  CDS (1)..(576)
atggaggatcaggatgctgcatttatcaaacaggctacagaagcaatagtggatgtatcattaaatatagataacatagatcctataataaaagagttattagaaagggtaaggaataggcaaaacaggttacaaaataaaaaaccagcactcataccggcagaaaatggtgttgatataaatagtcaaggcggtaacataaaggttaaaaaggaaaacgcattaccaaaaccaccgaagtccagcaaaagcaaaccccaagatcgtagaaatagtactggtgaaaaaagatttaaatgtgcgaaatgttcgttggaattttcaagatcatcagatttgagaaggcacgaaaagacacacttcgccatattgcctaacatttgtcctcaatgtggcaaaggttttgcaaggaaagatgcattgaaaagacattatgatacactgacatgtaggagaaacaggactaaattactaactgcgggtggtgagggtatcaatgaattactgaaaaaagtcaagcaatccaacatcgttcatcgtcaagataacaaccacaatggtagcagtaatggctga

SEQ ID NO: 22 - protein, 191 aa, Saccharomyces cerevisiae
MEDQDAAFIKQATEAIVDVSLNIDNIDPIIKELLERVRNRQNRLQNKKPALIPAENGVDINSQGGNIKVKKENALPKPPKSSKSKPQDRRNSTGEKRFKCAKCSLEFSRSSDLRRHEKTHFAILPNICPQCGKGFARKDALKRHYDTLTCRRNRTKLLTAGGEGINELLKKVKQSNIVHRQDNNHNGSSNG

SEQ ID NO: 23 - DNA, 33 bp, Artificial Sequence
  Description of Artificial Sequence  Synthetic primer
ccatcgatggcacagagcatagatgcgccgacc

SEQ ID NO: 24 - DNA, 30 bp, Artificial Sequence
  Description of Artificial Sequence  Synthetic primer
ggaattccgaaggtttgctgtattcggagc

SEQ ID NO: 25 - DNA, 29 bp, Artificial Sequence
  Description of Artificial Sequence  Synthetic primer
ggaattccccccttgctgtggcatgctag

SEQ ID NO: 26 - DNA, 32 bp, Artificial Sequence
  Description of Artificial Sequence  Synthetic primer
cgggatcccggtatagcgaaggttgttgtccc

SEQ ID NO: 27 - DNA, 66 bp, Artificial Sequence
  Description of Artificial Sequence  Synthetic primer
gctcttgtaaacgacgtcaaatcttcatatgcaaggagatctgattcctgacttcaactcaagacg

SEQ ID NO: 28 - DNA, 75 bp, Artificial Sequence
  Description of Artificial Sequence  Synthetic primer
ccgacggattcagaggtaaataagaaagttttgctcttggacattgttttatatttgttgtaaaaagtagataat

SEQ ID NO: 29 - DNA, 101 bp, Artificial Sequence
  Description of Artificial Sequence  Synthetic primer
tgatgttatgtcgagagctctgaaaaccaattattttgaaagctaacatttcaaaaggctatttcttctgaaatatcgattcctgacttcaactcaagacg

SEQ ID NO: 30 - DNA, 91 bp, Artificial Sequence
  Description of Artificial Sequence  Synthetic primer
gtgtaacatgctgcttttagcatatatataatgttaagaattatttaacttctttaaaaacgcatttacgttaaaattccaatttctttgg

SEQ ID NO: 31 - DNA, 69 bp, Artificial Sequence
  Description of Artificial Sequence  Synthetic primer
cacgtccagcggattgctgctccttagtagtccacagttcttaaggattcctgacttcaactcaagacg

SEQ ID NO: 32 - DNA, 76 bp, Artificial Sequence
  Description of Artificial Sequence  Synthetic primer
atttttcaagtctttcagttcgtatagagacttcgacgacgacattgttttatatttgttgtaaaaagtagataat

(19) United States
(12) Patent Application Publication
(10) Pub. No.: US 2010/0150871 A1
(43) Pub. Date: Jun. 17, 2010

(54) Genetically-modified strain of yeast with an increased production and output of s-adenosylmethionine (sam)

(75) Inventor: Dominique Thomas, Gif Sur Yvette (FR)

Correspondence Address: YOUNG & THOMPSON, 209 Madison Street, Suite 500, Alexandria, VA, 22314, US

(73) Assignee: CENTRE NATIONAL DE LA RECHERCHE SCIENTIFIQE, Paris Cedex 16 (FR)

(21) Appl. No.: 11/666,289

(22) PCT Filed: Oct. 26, 2005
(86) PCT No.: PCT/FR2005/002670

§ 371 Date: Nov. 24, 2009

(30) Foreign Application Priority Data
Oct. 27, 2004 (FR) 0411454

(51) Int. Cl. A61K 36/06; C12P 13/12; C12N 1/19

(52) U.S. Cl. 424/93.2; 435/113; 435/254.2

(57) ABSTRACT

The invention relates to a genetically-modified strain of yeast, in which the gene coding for adenosine kinase has been inactivated by genetic modification for the production of S-adenosylmethionine (SAM).

The present invention relates to a genetically modified yeast strain exhibiting an increased production and excretion of S-adenosylmethionine (SAM), its use for the production of SAM, and pharmaceutical compositions comprising it.

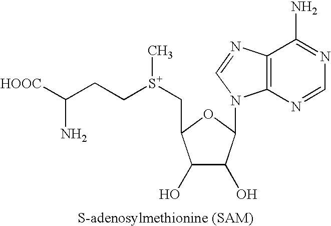

S-adenosylmethionine (SAM) (see the above formula) is an essential compound in the intermediate metabolism in all living organisms. This metabolite is synthesized from methionine and adenosine triphosphate (ATP). This synthesis, catalyzed by SAM synthetases (ATP: L-methionine S-adenosyltransferase, EC 2.5.1.6), can be considered as the terminal stage in the metabolism of sulphur-containing amino acids. This reaction is somewhat exceptional as it involves the total dephosphorylation of the ATP molecule used (Methionine+ATP→SAM+PPi+Pi) and therefore represents a particularly high energy consumption.

SAM is certainly the molecule which, after ATP, participates in the greatest number of cellular reactions. SAM in fact plays the role of a quasi-universal methyl group donor in the transmethylation reactions of proteins, nucleic acids, lipids etc. SAM also serves as a carboxy-aminopropyl group donor in reactions of modifications of bases of ribosomal RNAs or of transfers, or also during the synthesis of diphthamide, a post-translational modification of a histidine residue found in the elongation factor EF2 in eukaryotic organisms. SAM also serves as an amine group donor during biotin biosynthesis in microorganisms. SAM is also used as a precursor in the syntheses of polyamines, spermine and spermidine where, after an initial decarboxylation stage, it fulfils the role of an aminopropyl group donor (Cantoni, 1982, “The biochemistry of S-adenosylmethionine”, Columbia University Press, NY). This non-exhaustive description of the different metabolic functions of SAM demonstrates the central role played by this molecule in the intermediate metabolism.

In human medicine S-adenosylmethionine is indicated in the treatment of depression (SAMYR®). It has also been involved in the treatment of male sterility (Piacentino et al. (1991) Minerva Gynecol. 43:191-193), osteoarthritis (Najm et al. (2004) BMC Muscoskeletal Disord. 5:6-21) and fibromyalgia (Tavoni et al. (1998) Clin. Exp. Rheumatol. 16:106-107).

At present, no possibility exists of chemically synthesizing SAM with yields sufficient to obtain pharmaceutical-quality SAM at a cost compatible with marketing. The industrial SAM production processes involve microbiological production which is carried out with the yeast Saccharomyces cerevisiae.

Several documents of the prior art describe genetically modified yeast strains exhibiting increased production of SAM.

Thus, the patent application US 2002/0192784 relates to Saccharomyces cerevisiae yeast strains exhibiting increased production of SAM which have been genetically modified by the introduction of a chimeric gene coding for a methylene tetrahydrofolate reductase (MTHFR) composed partly of the Arabidopsis thaliana enzyme and partly of the yeast enzyme.

However, SAM does not appear to be excreted by these yeast strains, which makes the process for obtaining SAM from these strains of little interest from the point of view of industrial production, to the extent that the extraction of SAM from the yeast cells is a relatively lengthy and expensive process; by contrast, the purification of SAM from the culture medium of the yeast strains is particularly easy. Moreover, the yeast strains described in this document are genetically modified by the introduction of heterologous nucleic sequences, which poses a problem of biological safety, as the effects of these novel nucleic sequences and the proteins that they encode, in particular on human health, are not known.

The document Shiomi et al. (1995) Appl. Microbiol. Biotechnol. 42:730-733 describes a yeast strain Saccharomyces cerevisiae exhibiting increased production of SAM, genetically modified by the introduction of additional copies of an ethionine resistance gene.

The document Yu et al. (2003) Sheng Wu Hua Xue Yu Sheng Wu Wu Li Xue Bao 35:127-132 describes a yeast strain Pichia pastoris exhibiting increased production of SAM, genetically modified by the introduction of additional copies of the gene coding for S-adenosylmethionine synthetase 2 (SAM2).

SAM does not appear to be excreted by the yeast strains described in these two documents, moreover the production of SAM by these strains appears to be insufficient for an industrial application.

A subject of the present invention is therefore to provide genetically modified yeast strains exhibiting greater production and/or excretion of SAM than the yeast strains of the prior art.

Another subject of the invention is to provide genetically modified yeast strains not comprising heterologous nucleic sequences, i.e. not originating from an organism of a species different from that of the yeast considered.

Yet another subject of the invention is to use the genetically modified yeast strains of the invention for the industrial production of SAM.

Finally, another subject of the invention is to provide pharmaceutical compositions comprising the genetically modified yeast strains of the invention.

The present invention follows, in particular, from the demonstration by the Inventors that a yeast strain for which the ADO1 gene, encoding yeast adenosine kinase, was inactivated exhibited increased production and excretion of S-adenosylmethionine compared with the corresponding wild-type strain.

The present invention relates to the use of a genetically modified yeast strain, in which the gene coding for adenosine kinase has been inactivated by genetic modification, for the production of S-adenosylmethionine (SAM).

The term “yeast” is used to designate any unicellular-type fungus. The yeasts include in particular the genera Saccharomyces, Candida, Pichia, Schizosaccharomyces, and Kluyveromyces.

Adenosine kinase is also called adenosine 5 phosphotransferase.

In the yeast Saccaromyces cerevisiae, the gene coding for the adenosine kinase is the ADO1 gene corresponding to the open reading frame YJR105w (Lecoq et al. (2001) Yeast 18:335-342) and is in particular represented by SEQ ID NO: 1. Moreover, by way of example, for Kluyveromyces lactis, it is represented by the reference EMBL CR382124 and for Schizosaccharomyces pombe it is represented by the reference EMBL SPCC338.

In a particular embodiment, the invention therefore relates to the use of a genetically modified yeast strain, in which the ADO1 gene coding for adenosine kinase has been inactivated by genetic modification, for the production of S-adenosylmethionine (SAM).

A yeast gene is described as “inactivated” when the product for which it codes is not expressed in said yeast. In the case of the gene coding for adenosine kinase, this gene is described as inactivated if adenosine kinase is not expressed.

Numerous methods for inactivation of a given gene are known to a person skilled in the art, among which there can be mentioned:
the introduction of point mutations into the coding sequence of the gene in question, such as:
   nucleotide substitutions leading to false-sense (change of amino acid in the protein encoded by the gene to be inactivated) or non-sense (stop codon) mutations;
   insertions or deletions of nucleotides leading to an interruption of the open reading frame of said gene;
the insertion of nucleotide sequences coding for example for selection genes within the coding sequence of the gene to be inactivated;
the replacement of a substantial part of the coding sequence of the gene to be inactivated by nucleotide sequences coding for example for selection genes;
the modification of the promoter sequence of the gene to be inactivated, in particular at the level of consensus sequences, such as the TATA box;
the introduction into the yeast genome of sequences coding for an anti-sense of the messenger mRNA of the gene to be inactivated.

A selection gene is usually an antibiotic resistance gene (for example a geneticin resistance gene) or the wild-type copy of a gene coding for a stage of the yeast metabolism (for example URA3, TRP1, LEU2, or HIS3), which gene is mutated and inactive in the yeast which is to be modified.

All these methods are particularly easy for a person skilled in the art to implement, in particular when the sequence of the gene to be inactivated is known.

Advantageously, the inactivation of the gene coding for adenosine kinase in a yeast strain leads to the increased production and excretion of S-adenosylmethionine by said strain.

The determination of the sequence of the gene coding for adenosine kinase of the yeasts for which it has not been identified is easy for a person skilled in the art. In the case where the genome of the yeast for which the gene coding for adenosine kinase to be identified is partially or completely sequenced it is possible to launch a search in the databases of appropriate sequences, either on the basis of the name of the enzyme encoded by the sought gene, i.e. adenosine kinase, or by homology with a known sequence of the gene, for example ADO1, using software such as the BLAST software (Altschul et al. (1997) Nucleic Acids Res. 25:3389-3402). Alternatively, it is also possible to screen a cDNA or genomic DNA bank of the yeast in question using a probe derived from a known sequence of the gene, for example ADO1, as is well known to a person skilled in the art; to this end reference can be made, for example, to the work Sambrook and Russel (2000) Molecular Cloning: A Laboratory Manual, Cold Spring Harbor Laboratory Press.

According to a particular embodiment of the invention, the sequence of the gene coding for adenosine kinase has been genetically modified by mutation.

The term “mutation” is used to designate a given nucleotide sequence, the insertion, the substitution and/or the suppression of one or more nucleotides in said nucleotide sequence.

According to a preferred embodiment of the invention, the sequence of the gene coding for the adenosine kinase of the genetically modified strain defined above has been disrupted.

The term “disruption” of a gene sequence is used to designate the interruption of the coding sequence of said gene by the introduction of a nucleotide sequence within this coding sequence. In general, this introduction is accompanied by the substitution of a substantial part of the coding sequence, or even of the whole coding sequence, by the introduced sequence. This introduced sequence is usually a selection gene.

Numerous disruption methods are available to a person skilled in the art such as, for example, those described by Rothstein (1991) Methods Enzymol. 194:281-301; Wach et al. (1994) Yeast 10:1793-1808 or also Güldener et al. (1996) Nucleic Acids Res. 24:2519-254.

According to a particular embodiment of the invention, the genetically modified strain defined above has at least one other genetic modification chosen from the group comprising:
the inactivation of a gene chosen from the group comprising the gene coding for the high-affinity transporter of S-adenosylmethionine, the gene coding for S-adenosylmethionine-homocysteine methyl transferase, the gene coding for S-methylmethionine-homocysteine methyl transferase, and the gene coding for the Met30 receptor sub-unit of the ubiquitin ligase complex SCF^{Met30},
the introduction of an additional copy of the sequence of a gene chosen from the group comprising the gene coding for S-adenosylmethionine synthetase 1, the gene coding for S-adenosylmethionine synthetase 2, the gene coding for low-affinity methionine permease, the gene coding for high-affinity methionine permease, the gene coding for very low-affinity methionine permease, a gene coding for a broad-spectrum permease which can transport methionine, in the genome of said strain, and
the mutation of the promoter sequence of a gene chosen from the group comprising the gene coding for S-adenosylmethionine synthetase 1, the gene coding for S-adenosylmethionine synthetase 2, the gene coding for low-affinity methionine permease, the gene coding for high-affinity methionine permease, the gene coding for very low-affinity methionine permease, a gene coding for a broad-spectrum permease which can transport methionine.

The gene coding for the S-adenosylmethionine synthetase 1 of yeast is in particular represented by the SAM1 gene of Saccharomyces cerevisiae (Thomas and Surdin-Kerjan (1987) J. Biol. Chem., 262: 16704-16709).

The gene coding for the S-adenosylmethionine synthetase 2 of yeast is in particular represented by the SAM2 gene of Saccharomyces cerevisiae (Thomas et al. (1998) Mol. Cell. Biol. 8: 5132-5139).

The gene coding for the high-affinity transporter of S-adenosylmethionine is in particular represented by the SAM3 gene of Saccharomyces cerevisiae (Rouillon et al., J. Biol. Chem. 274: 28096-28105).

The gene coding for S-adenosylmethionine-homocysteine methyl transferase is in particular represented by the SAM4 gene of Saccharomyces cerevisiae (Thomas et al. (2000) J. Biol. Chem. 275: 40718-40724).

The gene coding for S-methylmethionine-homocysteine methyl transferase is in particular represented by the MHT1 gene of Saccharomyces cerevisiae (Thomas et al. (2000) J. Biol. Chem. 275: 40718-40724).

The gene coding for the Met30 receptor sub-unit of the ubiquitin ligase complex SCF^{Met30} in particular represented by the MET30 gene of Saccharomyces cerevisiae (Patton et al. (1998) Genes Dev. 12: 692-705).

The gene coding for low-affinity methionine permease is in particular represented by the AGP1 gene of Saccharomyces cerevisiae (Iraqui et al. (1999) Mol. Cell. Biol. 19: 989-1001).

The gene coding for high-affinity methionine permease is in particular represented by the MUP1 gene of Saccharomyces cerevisiae (Isnard et al. (1996) J. Mol. Biol. 262: 473-484).

The gene coding for the very low-affinity methionine permease is in particular represented by the MUP3 gene of Saccharomyces cerevisiae (Isnard et al. (1996) J. Mol. Biol. 262: 473-484).

A gene coding for a broad-spectrum permease capable of transporting methionine is in particular represented by the BAP2 gene or by the BAP3 gene of Saccharomyces cerevisiae (Regenberg et al. (1999) Curr. Genet. 36: 317-328).

According to a more particular embodiment of the invention, the genetically modified strain defined above has at least one other genetic modification chosen from the group comprising:
the inactivation of a gene chosen from the group comprising SAM3, SAM4, MHT1, and MET30,
the introduction of an additional copy of the sequence of a gene chosen from the group comprising SAM1, SAM2, AGP1, MUP1, MUP3, BAP2, and BAP3, into the genome of said strain, and
the mutation of the promoter sequence of a gene chosen from the group comprising SAM1, SAM2, AGP1, MUP1, MUP3, BAP2, and BAP3.

The sequence of the SAM1 gene of the yeast Saccharomyces cerevisiae (SEQ ID NO: 3) and of its translation product, the protein Sam1p (SEQ ID NO: 4), is that deposited in the EMBL database, identifier “SCSAM1”, accession number J03477.

The sequence of the SAM2 gene of the yeast Saccharomyces cerevisiae (SEQ ID NO: 5) and of its translation product, the protein Sam2p (SEQ ID NO: 6), is that deposited in the EMBL database, identifier “SCSAM2SA”, accession number M23368.

The sequence of the SAM3 gene of the yeast Saccharomyces cerevisiae (SEQ ID NO: 7) and of its translation product, the protein Sam3p (SEQ ID NO: 8), is that deposited in the EMBL database, identifier “SCYPL274W”, accession number Z73630.

The sequence of the gene SAM4 of the yeast Saccharomyces cerevisiae (SEQ ID NO: 9) and of its translation product, the protein Sam4p (SEQ ID NO: 10), is that deposited in the EMBL database, identifier “SCYPL273W”, accession number Z73629.

The sequence of the MHT1 gene of the yeast Saccharomyces cerevisiae (SEQ ID NO: 11) and of its translation product, the protein Mht1p (SEQ ID NO: 12), is that deposited in the EMBL database, identifier “SCYLL062C”, accession number Z73167.

The sequence of the AGP1 gene of the yeast Saccharomyces cerevisiae (SEQ ID NO: 13) and of its translation product, the protein Agp1p (SEQ ID NO: 14), corresponds to the sequence of ORF YCL025C and is comprised in the sequence deposited in the EMBL database, identifier “SCCHRIII”, accession number X59720.

The sequence of the BAP2 gene of the yeast Saccharomyces cerevisiae (SEQ ID NO: 15) and of its translation product, the protein Bap2p (SEQ ID NO: 16), is that deposited in the EMBL database, identifier “SCYBR068C”, accession number Z35937.

The sequence of the BAP3 gene of the yeast Saccharomyces cerevisiae (SEQ ID NO: 17) and of its translation product, the protein Bap3p (SEQ ID NO: 18), corresponds to the sequence of ORF YDR046C and is comprised in the sequence deposited in the EMBL database, identifier “SC9609X”, accession number Z49209.

The determination of the sequence of the genes described above for other species of yeast, is easy for a person skilled in the art, who can for example proceed as described previously for the gene coding for adenosine kinase.

Advantageously, the inactivation of the genes coding for the high-affinity transporter of S-adenosylmethionine, for S-adenosylmethionine-homocysteine methyl transferase, S-methylmethionine-homocysteine methyl transferase, and the Met30 receptor sub-unit of the ubiquitin ligase complex SCF^{Met30} in a yeast strain makes it possible to increase the production and/or excretion of SAM by said strain.

Moreover the increase in the transcription of the genes coding for S-adenosylmethionine synthetase 1, S-adenosylmethionine synthetase 2, low-affinity methionine permease, high-affinity methionine permease, very low-affinity methionine permease, a broad-spectrum permease which can transport methionine, either by modification of their promoter sequences, or by increasing their number of copies in a yeast strain, also makes it possible to increase the production and/or excretion of SAM by said strain.

When an additional copy of the sequence of a gene is introduced into the genome of the genetically modified strain defined above, this copy can be either carried by an autonomous replication vector such as a plasmid or an artificial chromosome, or be inserted into one or more chromosomes of said genetically modified strain.

In a particular embodiment of the invention, the genetically modified strain defined above carries a modified copy of the gene coding for methylene tetrathydrofolate reductase (MET13 for Saccharomyces cerevisiae, Raymond et al. (1999) Arch. Biochem. Biophys. 372: 300-308), the corresponding enzyme no longer being inhibited by SAM.

According to another particular embodiment of the invention, the sequence of at least one of the genes of the genetically modified strain defined above is chosen from the group comprising the gene coding for the high-affinity transporter of S-adenosylmethionine, the gene coding for S-adenosylmethionine-homocysteine methyl transferase, the gene coding for S-methylmethionine-homocysteine methyl transferase, and the gene coding for the Met30 receptor sub-unit of the ubiquitin ligase complex SCF^{Met30}, has been disrupted.

In another particular embodiment of the invention, at least one promoter sequence of one of the genes of the genetically modified strain defined above chosen from the group comprising the gene coding for S-adenosylmethionine synthetase 1, the gene coding for S-adenosylmethionine synthetase 2, the gene coding for low-affinity methionine permease, the gene coding for high-affinity methionine permease, the gene coding for very low-affinity methionine permease, a gene coding for a broad-spectrum permease which can transport methionine, has been substituted by a strong promoter sequence of yeast.

The expression “strong promoter” designates a promoter conferring upon the gene placed under its control, i.e. in general directly downstream of the promoter sequence, a higher transcription level than the average transcription level of the genes in the yeast. The strong promoters of yeasts are in particular described in Velculescu et al. (1997) Cell 88: 243-251.

These are in particular natural promoters of the PGK1, ADH1, TDH3, TEF1, PHO5, LEU2, and GAL1 genes.

The sequence of the promoter of the GAL1 gene of the yeast Saccharomyces cerevisiae used in the descriptions which follow corresponds in particular to the sequence described by Johnston and Davis (1984) Mol. Cell. Biol. 4: 1440-1448.

The sequence of the promoter of the TEF1 gene of the yeast Saccharomyces cerevisiae used in the descriptions which follow corresponds in particular to the sequence described by Schaaff-Gerstenschlager et al. (1993) Eur. J. Biochem. 217: 487-492).

The sequence of the promoter of the PGK1 gene of the yeast Saccharomyces cerevisiae used in the descriptions which follow corresponds in particular to the sequence described by Bonneau et al. (1991) Yeast 87: 609-615).

The sequence of the promoter of the PHO5 gene of the yeast Saccharomyces cerevisiae used in the descriptions which follow corresponds in particular to the sequence described by Feldmann et al. (1994) EMBO J. 13: 5795-5809).

Advantageously, the use of a strong promoter makes it possible to increase the transcription level of the genes placed under its control and therefore the translation level of the corresponding proteins, which induces an increase in the synthesis of SAM.

In a preferred embodiment of the invention, the genes coding for the Met30 receptor sub-unit of the ubiquitin ligase complex SCF^{Met30}, S-methylmethionine-homocysteine methyl transferase, adenosine kinase, and S-adenosylmethionine-homocysteine methyl transferase of the genetically modified strain defined above are inactivated, in particular by disruption of the sequences of said genes.

In another preferred embodiment of the invention, an additional copy of the sequence of the gene coding for S-adenosylmethionine synthetase 2, coupled with a strong promoter, has been introduced in the genome of the genetically modified strain defined above.

In another preferred embodiment of the invention, the sequence of the promoter of the gene coding for low-affinity methionine permease of the genetically modified strain defined above has been substituted by the strong promoter sequence.

In a particularly preferred embodiment of the invention, the gene coding for the high-affinity transporter of S-adenosylmethionine and the gene coding for the S-adenosylmethionine-homocysteine methyl transferase of said strain have been inactivated by substitution of the sequence of said genes by a copy of the sequence of the gene coding for S-adenosylmethionine synthetase 2, coupled with a strong promoter.

According to an advantageous embodiment of the invention, the strong promoter defined above is chosen from the group comprising the natural promoters of the PGK1, ADH1, TDH3, TEF1, PHO5, LEU2, GAL1 genes of the genetically modified strain defined above.

In another particularly preferred embodiment, the genetically modified strain defined above is prototrophic for adenine.

The expression “prototrophic for adenine” means that the strain defined above requires no exogenous supply of adenine for its growth and that it is capable of ensuring by itself the production of adenine necessary for its survival and/or growth. This means that the genes involved in the adenine synthesis route are functional. In particular, the strains defined above carry a wild-type, i.e. non-mutated, allele of the gene which codes for phosphoribosylaminoimidazole carboxylase (ADE2 in Saccharomyces cerevisiae).

In another particular embodiment of the invention, the genetically modified yeast strain defined above is haploid.

In an alternative embodiment of the invention, the genetically modified yeast strain defined above is diploid.

According to a particular embodiment of the invention, the genetically modified yeast strain according to the invention is characterized in that when the genetic modifications are chromosomic, said genetic modifications are carried by each of the two homologous chromosomes.

In a preferred embodiment of the invention, the genetically modified yeast strain according to the invention comprises no heterologous nucleotide sequences.

The term “heterologous” designates nucleotide sequences that are not found present in the natural state in the yeast cells belonging to the same species of yeast as that of the strain considered.

In another preferred embodiment of the invention, the genetically modified yeast strain according to the present invention is chosen from the group comprising the yeasts of the genera Saccharomyces, Candida, Pichia, Schizosaccharomyces, and Kluyveromyces, and said strain is in particular a yeast of the species Saccharomyces cerevisiae.

In a particular embodiment of the invention, the strain belongs to the species Saccharomyces cerevisiae and when the gene coding for the Met30 receptor sub-unit of the ubiquitin ligase complex SCF^{Met30} (MET30) of said strain is inactivated, in particular by disruption of the sequence of the MET30 gene, then the MET4 gene and/or the MET32 gene of said strain is also inactivated, in particular by disruption of the corresponding gene sequences.

The MET4 gene codes for the main transcriptional activator of the metabolism of the sulphur-containing amino acids in the yeast Saccharomyces cerevisiae (Thomas et al. (1992) Mol. Cell. Biol. 12: 1719-1727).

The MET32 gene codes for a transcriptional factor involved in the regulation of the metabolism of the sulphur-containing amino acids of the yeast Saccharomyces cerevisiae (Blaiseau et al. (1997) Mol. Cell. Biol. 17: 3640-3648).

In a wild-type yeast strain the inactivation of the MET30 gene is lethal for the strain in which it has been carried out. Advantageously the prior inactivation of MET4 and/or of MET32 makes it possible to eliminate the lethal effect of the inactivation of MET30 (Patton et al. (2000) EMBO J. 19: 1613-1624).

The sequence of the MET4 gene of the yeast Saccharomyces cerevisiae (SEQ ID NO: 19) and of its translation product, the protein Met4p (SEQ ID NO: 20), is that deposited in the EMBL database, identifier “SCMETBLZP”, accession number M84455.

The sequence of the MET32 gene of the yeast Saccharomyces cerevisiae (SEQ ID NO: 21) and of its translation product, the protein Met32p (SEQ ID NO: 22), is that deposited in the EMBL database, identifier “AY557729”, accession number AY557729.

The present invention also relates to a genetically modified yeast strain exhibiting increased production and excretion of S-adenosylmethionine compared with the corresponding non-modified yeast strain, said genetically modified strain being as defined above.

The present invention also relates to an S-adenosylmethionine production process, characterized in that it comprises the stages of:

culture of a genetically modified yeast strain as defined above in a culture medium,

purification of S-adenosylmethionine from the supernatant of the culture medium and/or from the genetically modified yeast cells.

Advantageously, the genetically modified yeast strains of the invention excrete a large quantity of SAM in their culture medium, which allows easy recovery of this product, without it being necessary to extract it from the yeasts, the cell wall of which is particularly resistant.

According to a particular embodiment of the process defined above, the culture is carried out in a chemostat.

Advantageously, a chemostat allows continuous culture of the yeasts. In fact, the term “chemostat” is used in particular to designate a bioreactor in which the supply of nutritive medium and the removal of the culture medium containing the yeasts are carried out continuously, in order to keep the physico-chemical parameters of the culture essentially constant while recovering the culture medium.

The present invention also relates to a pharmaceutical composition, characterized in that it comprises as active ingredient at least one yeast strain as defined above, in combination with a pharmaceutically acceptable vehicle.

Yeasts are known for being able to occur easily in the human intestine, the ingestion of cells of the yeast strains according to the invention, optionally freeze-dried, could thus make it possible to increase supplies of SAM in individuals needing them over a relatively long period, corresponding approximately to the duration of the colonization of the intestine by the yeasts.

The present invention also relates to the use of a genetically modified yeast strain as defined above, for the preparation of a medicament intended for the treatment of diseases requiring an increased supply of S-adenosylmethionine, such as depression, arthritis, fibromyalgia, or male sterility.

The present invention also relates to the use of a genetically modified yeast strain as defined above, for the preparation of foods or drinks enriched with S-adenosylmethionine.

The use of the genetically modified yeast strains of the invention for the preparation of certain foods or drinks (such as bread or beer in the case of Saccharomyces cerevisiae allows their spontaneous enrichment with SAM.

The present invention also relates to a food preparation or a drink, intended for human or animal consumption, characterized in that it comprises at least one genetically modified yeast strain as defined above.

### DESCRIPTION OF THE FIGURES

FIG. 1 diagrammatically represents the stages of cloning the MHT1 gene of Saccharomyces cerevisiae.

FIG. 2A diagrammatically represents the cloning of the SAM2 gene of Saccharomyces cerevisiae under the control of the strong promoter of the PGK1 gene.

FIG. 2B diagrammatically represents the stages of disruption of the SAM3 and SAM4 genes of Saccharomyces cerevisiae by the substitution of a copy of the SAM2 gene under the control of the promoter of the PGK1 gene.

FIG. 3 diagrammatically represents the cloning of the AGP1 gene of Saccharomyces cerevisiae under the control of the strong promoter of the PGK1 gene.

### EXAMPLES

### Methods and Materials

### Nomenclature Used

The nomenclature used in this work in order to designate the genes of Saccharomyces cerevisiae is the standard nomenclature known to a person skilled in the art: each gene is designated by three italic letters followed by a number, for example ADO1. The dominant alleles (in the present case, the wild-type allele) are denoted by italic capital letters, whilst the lower case letters are used in order to designate the recessive allele (in the present case, the mutated allele). The symbol ado1::URA3 designates the insertion of the gene URA3 at the ADO1 locus, in which URA3 is functional and ado1 is defective. The protein encoded by a given gene is designated by the name of the gene where the first letter is in upper case and the other two in lower case, followed by the letter “p”, for example Ado1p for the product of the ADO1 gene.

### Strains Used

The work described hereafter was carried out with haploid and auxotrophic strains of Saccharomyces cerevisiae for several amino acids or bases. These strains are isogenic with the strains:
CC788-2B (MATa, his3, leu2, ura3, trp1), described in Cherest et al. (2000) J. Biol. Chem. 275: 14056-14063, and
CC788-2D (MATa, his3, leu2, ura3, trp1) described in Cherest et al. (2000) J. Biol. Chem. 275: 14056-14063.

These two strains are themselves ADE2+ derivatives (prototrophic for adenine) of the reference strain W303 of the yeast Saccharomyces cerevisiae described by Bailis A. M. et al., 1990, Genetics 126, 535-547). The strain W303 is deposited at the ATCC under number ATCC 201239.

### Genetic Methods Used

The methods used for crossing strains and for dissecting the asci are those described by Sherman et al., 1979, “Methods in Yeast Genetics: a Laboratory Manual”, Cold Spring Harbor, N.Y.

The standard protocols for gene manipulation in Saccharomyces cerevisiae were used and the yeast transformations were carried out according to the method of Gietz et al., 1992, Nucleic Acids Res. 20, 1425. The gene replacements were carried out according to the method described by Rothstein (Rothstein, 1991, Methods Enzymol. 194, 281-301). The correct integration is verified each time either by “Southern blotting”, or by polymerase chain amplification (PCR).

The strategies used during gene manipulations were designed so that no E. coli DNA fragment remains in the yeast genome. The strains obtained therefore contain only Saccharomyces cerevisiae DNA sequences.

### Measurement of the Accumulation and Excretion of SAM by High-Pressure Chromatography

The measurements of SAM concentration in the culture supernatants or on cell extracts by high-pressure liquid chromatography were carried out according to the method of Wise et al., 1997, J. Chromatogr. B. Biomed. Sci. Appl., 696, 145-152) on a Waters Spherisob ODS2 resin, using of the standards of SAM of known concentrations.

### Measurement of the Excretion of SAM by a Microbiological Method.

For this work, a rapid method of measuring SAM by “cross-feeding” was developed. This method uses a test strain, which is a particular strain of Saccharomyces cerevisiae of genotype MATa, his3, leu2, ura3, ade2, trp, sam1::LEU2, sam2::HIS3 which exhibits a disruption of the two SAM1 and SAM2 genes encoding SAM synthetases and the growth of which absolutely requires an exogenous supply of SAM. In order to measure the excretion of SAM, this strain is cultured in medium containing increasing concentrations of supernatant of the cultures of the strains tested. The yield of the cultures thus produced is proportional to the quantity of SAM present in the supernatant. A calibration curve is produced in each experiment using SAM. The strain to be tested is cultured for 24 hours in 5 ml of complete YPD medium (Kuras et al. 2002, 2002, Mol. Cell 10, 69-80). After centrifugation, all of the cells are transferred to 5 ml of production medium and incubated under stirring for 24 hours at 30° C. The culture supernatants are then recovered by elimination of the cells by centrifugation followed by a filtration on a 0.45 micron filter and aliquots of these supernatants are added to cultures of the test strain.

### Example 1

### Identification of Genes the Inactivation of which Makes it Possible to Obtain Yeast Strains Producing and Excreting Increased Quantities of S-Adenosylmethionine (SAM)

### Disruption of the ADO1 Gene

In order to characterize novel strains capable of producing large concentrations of organic sulphur-containing compounds and in particular of SAM, the Inventors researched yeast strains the growth of which would be significantly altered by the presence in the culture medium of large quantities of SAM or one of its precursors, methionine or cysteine. A wild-type haploid strain of Saccharomyces cerevisiae (W303-1A) was mutagenized using ethyl methane sulphonate and colonies exhibiting growth defects on strong concentrations of organic sulphur-containing compounds, but still capable of growing on weak concentrations of these same compounds, were researched.

This work has made it possible in particular to characterize two genetically independent point mutations, provisionally named cys5-1 and cys6-1. The presence of each of these mutations induces a phenomenon of growth cessation when these cells are cultured in minimum medium containing 5 mM of L-cysteine. By contrast the mutated strains cys5-1 and cys6-1 are capable of growing in the presence of 0.2 mM L-cysteine used as a source of sulphur. The latter phenotype therefore demonstrates that the cys5-1 and cys6-1 mutations are different from the str1 and str4 mutations (also named cys1 and cys4 respectively) which are the only two mutations known at present which lead to a phenotype of non-growth on cysteine (Cherest and Surdin-Kerjan, 1992, Genetics 130, 51-58).

In order to understand the molecular nature of these mutations, the genes corresponding to these mutations were cloned by functional complementation. A genomic library constituted by fragments of DNA obtained by partial HindIII digestion of the DNA of a wild-type strain X2180-1A, cloned in the shuttle vector pEMBLYe23 (Baldari and Cesarini, 1985, Gene 35, 27-32) was employed and used in order to research plasmids capable of restoring the growth on 5 mM cysteine of the mutant strains cys5-1 and cys6-1. These experiments have made it possible to isolate plasmids comprising respectively a fragment of DNA comprising the open reading frame YER043w, situated on the right arm of the V chromosome of the yeast and complementing the growth defect of the strain cys5-1, and a fragment of DNA comprising the open frame YJR105w, situated on the right arm of the chromosome X and complementing the growth defect of the strain cys6-1. In order to confirm the identity of the CYS5 and CYS6 genes with respectively, the open frames YER043w and YJR105w, an integration technique directed at the locus was used and has confirmed these assignations.

Analysis of the peptide sequences deduced from these two open frames showed that:

i) the CYS5 gene (YER043w) corresponds to the gene specifying S-adenosylhomocysteine (SAH) hydrolase and named SAH1. The inactivation of this gene in a diploid strain W303 and the analysis of spores originating from the sporulation of the resultant diploid (sah1::URA3/SAH1) demonstrated that only two spores per tetrad were viable and that all the viable spores were auxotrophic for uracil, which means that the inactivation of the SAH1 gene is lethal in a haploid W303 gene context.

ii) the CYS6 gene (YJR105w) is equivalent to the ADO1 gene and codes for adenosine kinase (Lecoq et al., 2001, Yeast 18, 335-342). The ADO1 gene was inactivated in a diploid W303 and analysis of spores originating from the sporulation of the resultant diploid (ado1::URA3/ADO1) has shown that half of the 40 spores studied were incapable of growing on minimum medium containing 5 mM L-cysteine. All of the spores incapable of growing on cysteine 5 mM are protrophic for uracil and therefore correspond to spores comprising an inactivated allele of the ADO1 gene. These experiments demonstrate that the disruption of the ADO1 gene results in the same phenotype as the cells carrying the originally isolated cys6-1 mutation.

The ADO1 gene was cloned by functional complementation of a strain carrying a mutation in the ADO1 gene. The bank used was constructed by inserting the product of partial digestion by the HindIII restriction enzyme of the chromosomic DNA isolated from the strain X2180-1A into the HindIII site of the plasmid pEMBLYe23 (1) Baldari and Cesarini, 1985, Gene 35, 27-32). The plasmid thus isolated by functional complementation carried a 1690 by fragment containing the ADO1 gene deleted from the first 176 base pairs of the open reading frame. The plasmid carrying the ADO1 region was digested by the restriction enzyme EcoRI and auto-ligated, thus leading to the elimination of the 2-micron fragment from the plasmid pEMBLYe23 (Baldari and Cesarini (1985) Gene 35:27-32) and therefore to a plasmid incapable of being replicated in yeast. The fragment Asp718-Asp718 (460 bp) internal to the open reading frame was then replaced by the URA3 gene selection. This leads to the elimination of a fragment of Ado1p limited by the codons 118 and 271. The transformation of the CC572 diploid strain (originating from the W303-1A×W303-1B cross) by the HindIII-HindIII fragment of the plasmid thus obtained and selection of the transformants prototrophic for uracil, leads to the replacement of one of the chromosomic copies of the ADO1 gene by the one-stage disruption method (Rothstein, 1991, Methods in Enzymology, 194, 281-301). Analysis of the progeny of the diploid strain thus obtained (CD215) heterozygotic for the ado1::URA3 mutation shows that this disruption is not lethal. The disruption was verified by PCR.

The cloning of the genes corresponding to the cys5-1 and cys6-1 mutations therefore demonstrates that these two mutations affect two enzymes involved in the catabolism of SAM and more particularly involved in the catabolism of SAH, a product released during the methylation reactions using SAM. The mutations which affect the SAH1 or ADO1 genes therefore compromise the methyl cycle by leading to an accumulation of SAH, a molecule which is known to inhibit the methyltransferases which are the main users of SAM. These mutations are consequently capable of modifying the yield of the reactions which allow the synthesis of cysteine from methionine or cysteine (Thomas and Surdin-Kerjan, 1997, Microbiol. Mol. Biol. Rev. 61, 503-532).

The ability of strains carrying a mutation inactivating the ADO1 gene to be excreted from SAM was then analyzed.

The ADO1 gene was therefore disrupted in the CC788-2B and CC788-2D strains, as described above, in order to produce the CY78-3B and CY78-8B strains of genotypes MATa, his3, leu2, ura3, trp1, ado1::URA3 and MATa, his3, leu2, ura3, trp1, ado1::URA3, respectively.

These strains for which the ADO1 gene was disrupted produce approximately 10 times more, and excrete approximately 40 times more SAM, than their respective parental strains (see Table 1).

### Example 2

### Obtaining Strains Exhibiting a Reduction in the Catabolism of SAM

### Disruption of the SAM4 and MHT1 Genes

A reduction in the catabolism of SAM was therefore obtained by inactivating the ADO1 gene, which modifies the functions of the methyl cycle, this reduction of the catabolism of SAM being accompanied by an overproduction and increased excretion of SAM. It is however important to note that the methyl cycle is not the only SAM recycling route. In fact it has been recently demonstrated that the recycling of SAM can also take place via a homocysteine methylation reaction using SAM as methyl group donor. The existence of this second cycle was demonstrated during the functional characterization of two new genes, SAM4 and MHT1 which encode SAM-homocysteine methyltransferase and S-methylmethionine-homocysteine methyltransferase (Thomas et al., 2000, J. Biol. Chem. 275, 40718-40724) respectively. Whereas the enzyme Sam4p is very specific and seems to use only SAM as methyl donor substrate, the enzyme Mht1p uses S-methylmethionine and SAM as methyl donor substrate, but its affinity for this second substrate is 10 times weaker than for S-methylmethionine (Thomas et al., 2000, J. Biol. Chem. 275, 40718-40724). In order to increase the production of SAM by the yeast strains, mutations inactivating the SAM4 and MHT1 genes were introduced into the CY78-3B and CY78-8B strains of genotype MATa/a, his3, leu2, ura3, trp1, ado1::URA3 of Example 1.

### Cloning and Disruption of the MHT1 Gene

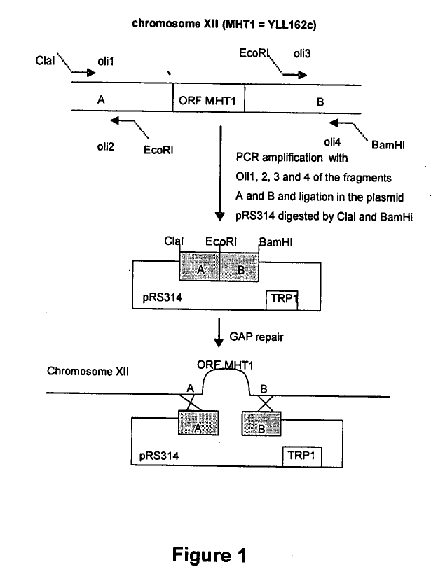

The MHT1 gene was cloned by the “GAP repair” method (Rothstein (1991) Methods Enzymol. 194: 281-301; Mallet and Jacquet (1996) Yeast 12: 1351-1357; see FIG. 1)

The DNA fragments comprised in the 5′ (A) and in 3′ (B) regions of the MHT1 gene were amplified by PCR using the oligonucleotides carrying Cla1 and EcoR1 (A) or EcoRI and BamHI (B) sites.

Oli1:

| | |
|---|---|
| CCATCGATGGCACAGAGCATAGATGCGCCGACC | (SEQ ID NO: 23) |

(ClaI site in bold type)
Oli2:

| | |
|---|---|
| GGAATTCCGAAGGTTTGCTGTATTCGGAGC | (SEQ ID NO: 24) |

(EcoRI site in bold type)
Oli3:

| | |
|---|---|
| GGAATTCCCCCCTTGCTGTGGATGCTAG | (SEQ ID NO: 25) |

(EcoRI site in bold type)
Oli4:

| | |
|---|---|
| CGGGATCCCGGTATAGCGAAGGTTGTTGTCCC | (SEQ ID NO: 26) |
| (BamHI site in bold type) | |

The amplification leads to the obtaining of 232 by (A) and 344 by (B) fragments, which were digested by EcoRI and by either ClaI (A), or BamHI (B). The fragments thus digested are ligated with the plasmid pRS314 digested by ClaI and BamHI, as shown by the figure above. The plasmid thus obtained, opened by EcoRI was then used for a “GAP repair” experiment with the chromosomic gene in the strain W303-1A.

In order to disrupt the MHT1 gene, the NcoI-HpaI fragment of the plasmid pRS314-MHT1 was replaced by a BamHI-BamHI fragment carrying the HIS3 selection gene of Saccharomyces cerevisiae (pRS314: Sikorski and Hieter (1989) Genetics 122:19-27; The sequence of the plasmid pRS314 is that deposited in the EMBL database, identifier “PRS314”, accession number U03440). This leads to the elimination from the codon 128 to the codon 236 of Mht1p. Then the plasmid was digested by ClaI and BamHI and the fragment obtained was used for a one-stage disruption by transforming the CY251-17D strain (Mat-a, his3, leu2, ura3, trp1, met32::TRP1, ado1::URA3, sam4::proSAM4-SAM2, and by selecting the transformants prototrophic for histidine. The disruption was verified by PCR using primers complementary to sequences adjacent to ORF MET32.

The SAM4 gene was cloned and inactivated as described in Thomas et al. (2000) J. Biol. Chem. 275: 40718-40724.

The mutant triple strains MATa/a, his3, leu2, ura3, trp1, ado1::URA3, mht1::HIS3, sam4::URA3 obtained are therefore totally deficient for the catabolism of SAM associated with its use as methyl group donor. As is seen in Table 1, the simultaneous presence of these three mutations significantly increases the quantity of SAM produced and excreted by the yeast cells.

The CY168-14A and CY168-1C strains were obtained by crossing the strains CY78-3B (Mata, his3, leu2, ura3, trp1, ado1::URA3) and CY55-5A (Matα, his3, leu3, ura3, ade2, trp1, sam4::URA3, mht1::HIS3) and by sporulating the diploids obtained, then by analyzing the tetrads obtained in order to select the spores having the desired gene recombinations. In such a cross, spores are obtained carrying the double disruption ado1::URA3, sam4::URA3 by selecting specifically the URA+ spores in the tetrads which have 2 URA⁺ spores and 2 URA⁻ spores (i.e. the recombinant ditype, as opposed to the tetratype tetrads, 3 URA⁺, 1 URA⁻).

### Example 3

### Obtaining Strains in which the Negative Transcriptional Regulation Acting on SAM Synthesis was Inactivated

### Disruption of the MET30 Gene

The synthesis of the organic sulphur-containing compounds, methionine, cysteine and SAM is subjected to a specific negative regulation, which acts at the transcriptional level and which is triggered following an increase in the intracellular concentration of organic sulphur-containing compounds. In order to significantly increase SAM synthesis by increasing the reduced flow of sulphur necessary for the synthesis of this compound, this negative regulation was suppressed and the genetic modifications necessary for this suppression were combined in the same strain with the mutations inactivating the catabolism of SAM and the transport of SAM described above.

It was possible to obtain the modification of the negative regulation of the biosynthesis of SAM by inactivating the MET30 gene which codes for the Met30 receptor sub-unit of the ubiquitin ligase complex SCF^{Met30} (Patton et al., 1998, Genes Dev., 12, 692-705). In order to carry out this inactivation, it was necessary to use strains first possessing an inactivated MET4 gene or an inactivated MET32 gene. In fact, the protein Met30p is an enzyme essential for maintaining yeast-cell viability and the inactivation of the MET30 gene is lethal. However, it was shown that the protein Met30p is not indispensable to yeast cells if the latter do not at the same time express either the protein Met4p or the protein Met32p (Patton et al., 2000, The Embo J. 19, 1613-1624).

### Disruption of the MET30 Gene

A BamHI-BamHI DNA fragment carrying the MET30 gene was cloned in the plasmid pUC19 as described in Thomas et al. (1995) Mol. Cell. Biol. 15: 6526-6534. The resultant plasmid was digested by EcoRV and dephoshorylated. A BglII-BglII fragment carrying the URA3 gene the ends of which have been freed is ligated with the plasmid pUC19 digested by EcoRV-dephoshorylated, producing a plasmid carrying the MET30 gene disrupted by the URA3 gene. This results in the elimination of codons 264 to 448 of Met30p. The diploid strain W303 was then transformed by the BamHI-SalI fragment and the transformants prototrophic for uracil were selected (one-stage disruption, Rothstein, 1991, Methods in Enzymology, 194, 281-301). Homologous recombination has thus led to the interruption of one of the chromosomic copies of the MET30 gene by the URA3 gene. This was verified by “Southern Blotting” using a radiolabelled probe corresponding to the URA3 gene. Analysis of the progeny of the diploid thus obtained (CD126), heterozygotic for the met30::URA3 mutation showed that the tetrads contain only two viable spores as shown previously (Thomas et al., 1995, Mol. Cell. Biol., 15, 6526-6534). It was subsequently demonstrated that the strains carrying the two mutations met30Δ, met4Δ or met30Δ, met32Δ are viable (Patton et al., 2000, The Embo J. 19, 1613-1624).

### Disruption of the MET32 Gene.

A HindIII-EcoRI DNA fragment carrying the MET32 gene was cloned in the plasmid pUC9. The TRP1 gene of Saccharomyces cerevisiae was then inserted into the only SwaI site present in the open reading frame of the MET32 gene (Blaiseau et al., (1997) Mol. Cell. Biol. 17: 3640-3648). The plasmid thus modified was digested by HindIII and EcoRI and the fragment obtained (3680 bp) was used for a one-stage disruption (Rothstein, 1991, Methods in Enzymology, 194, 281-301) transforming the strain W303-1A and selecting the transformants prototrophic for tryptophan. The disruption was verified by polymerase chain amplification (PCR).

The analyses carried out with the CY82-7C strains (genotype of interest; met32Δ, ado1Δ), CY117-3A (genotype of interest; met30Δ, met4Δ, ado1Δ) and CY154-17A (genotype of interest; met30Δ, met32Δ, ado1Δ) demonstrate that strains carrying a double inactivation of the MET4 and MET30 genes combined with inactivation of the ADO1 gene overproduce SAM in comparison with a wild-type strain. Moreover, the same analysis shows that the cells carrying a double inactivation of the MET30 and MET32 genes combined with inactivation of the ADO1 gene produce more SAM than the cells possessing a single inactivation of the ADO1 gene (see Table I).

The strain CY82-7C was obtained by growing the strains CC867-1D (Matα, his3, leu2, ura3, ade2, trp1, met32::TRP1, met30::URA3) and CY78-3B (Mata, his3, leu2, ura3, trp1, ado1::URA3) and sporulating the diploids obtained, then analyzing the tetrads obtained in order to select the spores having the desired gene recombinations.

The strain CY154-17A was obtained by growing the strains CC867-1D (Matα, his3, leu2, ura3, ade2, trp1, met32::TRP1, met30::URA3) and CY82-7C (Mata, his3, leu2, ura3, trp1, ado1::URA3, met32::TRP1).

This double inactivation of the MET30 and MET32 genes therefore makes it possible to inactivate the negative transcriptional regulation of the metabolism of the inorganic sulphur without the strains which possess this double mutation having a phenotype of specific auxotrophy. This is to be compared with the presence of a double mutation met4Δ, met30Δ which, if it allows the maintenance of the cell viability, results in a phenotype auxotrophic for methionine. The simultaneous inactivation of the MET30 and MET32 genes is therefore compatible with the growth of the strains on economically advantageous culture media.

### Example 4

### Obtaining Strains Showing an Increase in Sam Synthetase Activity

### Overexpression of the SAM2 Gene

As indicated in the introduction, one of the limiting stages of the biosynthesis of SAM is the reaction which allows the synthesis of this compound from methionine and ATP. In fact, this reaction is particular as it requires the total dephosphorylation of an ATP molecule. In yeast, two isoenzymes encoded by the SAM1 and SAM2 genes catalyze this reaction. The enzymatic mechanism of the reaction catalyzed by the SAM synthetases is a two-stage mechanism: the ATP and methionine molecules react in order to form SAM and triphosphate, then in a second stage, the triphosphate is cleaved to Pi and PPi. In order to increase the synthesis of SAM, the Inventors have sought to overexpress the SAM1 and SAM2 genes from different strong promoters of yeast, such as the promoters of the PGK1 and TEF1 genes.

Strains have been constructed in which the endogenous copies of the SAM1 and SAM2 genes have been replaced by the proPGK1-SAM1 and proPGK1-SAM2 fusions respectively or proTEF1-SAM1 and proTEF1-SAM2. Analysis of the SAM production of the corresponding strains has made it possible to show that these gene replacements lead to a slight increase in SAM production. An alternative strategy was therefore used and led to the addition of an additional copy of the SAM2 gene, which was inserted into the XVI chromosome of the yeast, in place of the two adjacent genes SAM3 and SAM4. Analysis of the production of SAM in such strains revealed that the presence of such an additional copy of the SAM2 gene at this gene locus induced a significant increase in the production of SAM by the yeast cells.

Construction of the proPGK1-SAM2 Fusion Gene

The SAM2 gene was placed under the control of the PGK1 gene promoter by a “GAP repair” experiment. For this purpose, the PGK1 gene promoter carried by the plasmid pFL61 (Bonneau et al. (1991) Yeast 87: 609-615) was amplified using two bi-functional oligonucleotides. Oli-5′-SAM2-PGK comprises 42 nucleotides homologous to the target sequence situated in the 5′ region of the SAM2 gene (−222 to −181, in bold type below), followed by 24 nucleotides homologous to the PGK1 gene promoter (−771 to −747). The second oligonucleotide (Oli-3′-SAM2-PGK) is formed by 45 nucleotides homologous to the SAM2 gene comprising the ATG initiating the translation (44 to 1, in bold type below) followed by 30 homologous nucleotides of the PGK1 gene promoter (−2 to −31). The nucleotides are counted from the A of the ATGs initiating the translation of each gene.

Oli-5′SAM2-PGK:

(SEQ ID NO: 27)

GCTCTTGTAAACGACGTCAAATCTTCATATGCAAGGAGATCTGATTCCTG
ACTTCAACTCAAGACG
Oli-3′-SAM2-PGK:

(SEQ ID NO: 28)

CCGACGGATTCAGAGGTAAATAAGAAAGTTTTGCTCTTGGACATTGTTTT
ATATTTGTTGTAAAAAGTAGATAAT

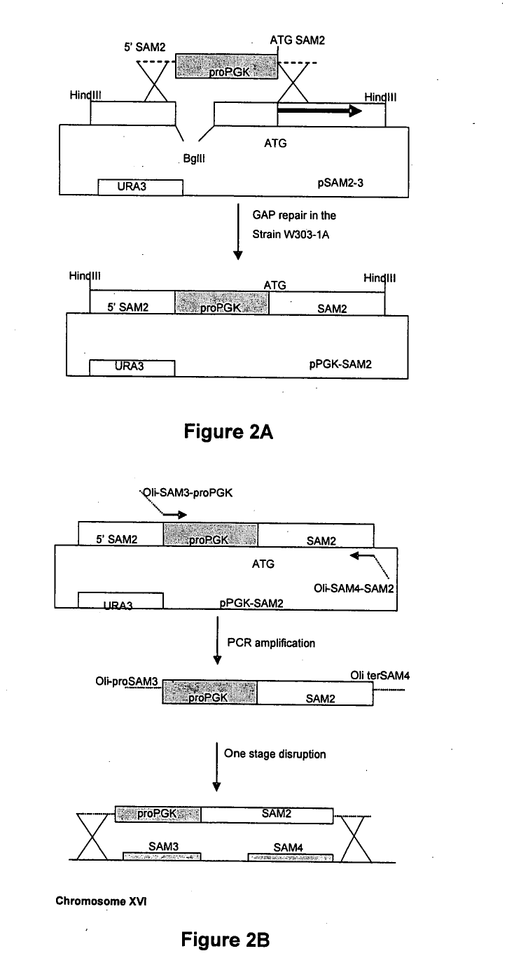

The fragment obtained by amplification is used in a “GAP repair” experiment with the plasmid pSAM2-3 (Thomas et al., 1988, Mol. Cell. Biol., 8, 5132-5139) in the strain W303-1A. The plasmid resulting from this experiment carries the SAM2 gene under the control of the PGK1 gene promoter (see FIG. 2A).

The plasmid obtained was digested by HindIII, and the fragment generated was used in a one-stage disruption experiment (Rothstein, 1991, Methods in Enzymology, 194, 281-301) by transforming the strain W744-1A and selecting transformants prototrophic for SAM. The disruption was verified by PCR.

Replacement of the SAM3 and SAM4 Genes by the proPGK1-SAM2 Fusion Gene

In an advantageous method of implementation of the processes according to the invention, the SAM4 gene was inactivated simultaneously with the adjacent SAM3 gene which codes for the high-affinity SAM transporter, in order to significantly reduce the possibility of the yeast cells again taking up the SAM that they would have excreted.

In another advantageous method of implementation of the processes according to the invention, this double inactivation of the SAM3 and SAM4 genes was carried out by inserting at their locus a fusion overexpressing the enzyme Sam2p (SAM synthetase 2) under the control of the strong constitutive promoter PGK1. The yeast cells thus constructed have the advantage of having, thanks to this specific modification made to their genome, a reduced SAM catabolism, a high affinity transport system of inactivated SAM and an increased expression of the SAM synthetase activity thanks to the presence of an additional SAM2 gene, of overgrowth expressed starting with a strong constitutive promoter (whereas the promoters of the native genes SAM1 and SAM2 are regulated negatively by the increase in the endogenous concentrations of organic sulphur-containing compounds, Thomas and Surdin-Kerjan, 1997, Microbiol. Mol. Biol. Rev. 61, 503-532). The strains carrying at the level of the left arm of the chromosome XVI the insertion of the proPGK1-SAM2 fusion in place of the SAM3 and SAM4 genes (construction indicated in the genotype of the strains such as sam3-sam4::proPGK1-SAM2) effectively demonstrate an increased synthesis of SAM compared with their parental strain.

The plasmid carrying the SAM2 gene placed under the control of the PGK1 gene promoter, obtained as described above, was used in order to amplify the fragment carrying the proPGK-SAM2 gene with bi-functional oligonucleotides. The oligonucleotide SAM3-PGK comprises 77 nucleotides homologous to the target sequence in the 5′ region of the SAM3 gene (−80 to −3, appearing in bold type below) followed by 24 nucleotides homologous to the PGK1 gene promoter region (−771 to −748). The oligonucleotide SAM4-SAM2 comprises 70 nucleotides homologous to the target sequence in the 3′ region of the SAM4 gene (1043 to 974, appearing in bold type below) followed by 21 nucleotides homologous to the end of the SAM2 gene (1155 to 1134). The nucleotides are numbered from the ATG initiating the translation for each gene.

Oli-SAM3-proPGK:

(SEQ ID NO: 29)

TGATGTTATGTCGAGAGCTCTGAAAACCAATTATTTTGAAAGCTAACATT
TCAAAAGGCTATTTCTTCTGAAATATCGATTCCTGACTTCAACTCAAGAC
G
Oli-SAM4-SAM2:

(SEQ ID NO: 30)

GTGTAACATGCTGCTTTTAGCATATATATAATGTTAAGAATTATTTAACT
TCTTTAAAAACGCATTTACGTTAAAATTCCAATTTCTTTGG

The fragment obtained by amplification was used in a one-stage disruption experiment (Rothstein, 1991, Methods in Enzymology, 194, 281-301) by transforming the W744-1A strain and selecting the transformants prototrophic for SAM (see FIG. 2B).

The simultaneous replacement of the SAM3 and SAM4 genes by the proPGK-SAM2 gene was verified by PCR.

The CY273 strains were obtained by crossing the strains CY270-7D (Mata, his3, leu2, ura3, trp1, met32::TRP1, ado1::URA3, sam3-sam4::proPGK1-SAM2) and CY258-10D (Matα, his3, leu2, ura3, trp1, met32::TRP1, met30::URA3, ado1::URA3, sam3-sam4::proSAM4-SAM2, mht1) and sporulating the diploids obtained, then analyzing the tetrads obtained in order to select the spores having the desired gene recombinations.

### Example 5

### Obtaining Strains Exhibiting Increased Methionine Transport Capacities

### Overexpression of the Gene AGP1

One of the processes implemented by the present invention relates to the culture of genetically modified yeast cells in order to produce large quantities of SAM. In an advantageous embodiment of the processes implemented, the culture medium of the yeast cells contains a high concentration of methionine, an inexpensive product, which is the immediate precursor of SAM. In order to increase the production of SAM under such culture conditions, the methionine transport capacity of the cells of the culture medium towards their cytoplasm was improved. In order to remove the methionine from the external medium, the yeast possesses at least three transport systems the enzymatic characteristics of which are different. These transport systems differ in particular by their affinity for methionine and their capacity to transport this sulphur-containing amino acid. These systems are called high-affinity methionine permease, encoded by the MUP1 gene, low-affinity methionine permease, encoded by the AGP1 gene (and perhaps other genes) and very low-affinity methionine permease, encoded by the MUP3 gene (Isnard et al., 1996, J. Mol. Biol. 262, 473-484) The expression of these different genes is subject to complex regulations and the methionine transport capacities therefore vary widely according to the growth conditions. In order to increase the removal of the methionine from the external medium and make it less dependent on environmental variations, yeast strains have been constructed in which the AGP1 gene coding for low-affinity methionine permease was placed under the control of the strong constitutive promoter PGK1. The proPGK1-AGP1 fusion was used in order to replace the endogenous copy of the AGP1 gene. The agp1::proPGK1-AGP1 mutation was introduced into strains exhibiting mutations met30Δ, met32Δ, sam4Δ, mht1Δ, ado1Δ and analysis of the corresponding CY208-7B strain demonstrates an increase in the quantity of SAM produced following the introduction at the AGP1 locus of the proPGK1-AGP1 fusion.

Replacement of the AGP1 Gene by the proPGK1-AGP1 Fusion Gene

The AGP1 gene was placed under the control of the PGK1 gene promoter by a “GAP repair” experiment. For this purpose, the PGK1 gene promoter carried by the plasmid pFL61 was amplified using two bi-functional oligonucleotides. Oli-5′-AGP1-PGK comprises 45 nucleotides homologous to the target sequence situated in the 5′ region of the AGP1 gene (−387 to −342, in bold type below), followed by 24 nucleotides homologous to the PGK1 gene promoter (−771 to −747). The second oligonucleotide (Oli-3′-AGP1-PGK) is formed by 45 nucleotides homologous to the AGP1 gene comprising the ATG initiating the translation (45 to 1, in bold type below) followed by 30 homologous nucleotides of the PGK1 gene promoter (−1 to −30). The nucleotides are counted starting from the A of the ATGs initiating the translation of each gene.

Oli-5′-AGP1-PGK:

(SEQ ID NO: 31)

CACGTCCAGCGGATTGCTGCTCCTTAGTAGTCCACAGTTCTTAAGGATTC
CTGACTTCAACTCAAGACG
Oli-3′-AGP1-PGK:

(SEQ ID NO: 32)

ATTTTTCAAGTCTTTCAGTTCGTATAGAGACTTCGACGACGACATTGTTT
TATATTTGTTGTAAAAAGTAGATAAT

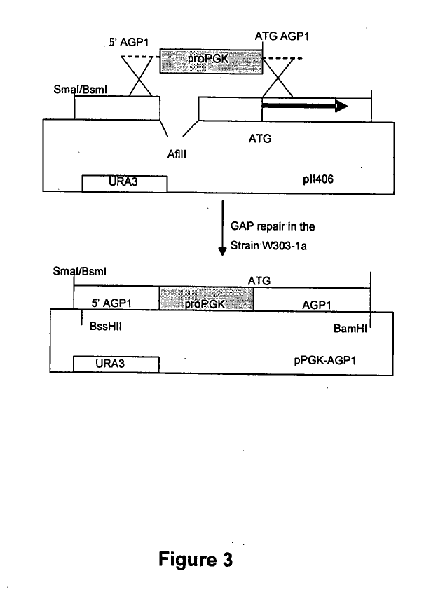

The fragment obtained by amplification was then used in a “GAP repair” experiment with the plasmid pII406 (the vector pFL38 carrying a 3 kb fragment comprising the AGP1 gene, Iraqui et al., 1999, Mol. Cell. Biol., 19, 989-1001) in the strain W303-1A. The plasmid resulting from this experiment carries the AGP1 gene under the control of the PGK1 gene promoter (see FIG. 3).

This plasmid was digested by BssHII and BamHI, and the fragment generated was used in a one-stage disruption experiment (Rothstein, 1991, Methods in Enzymology, 194, 281-301), by transforming the strain EK011 (Iraqui et al. (1999) Mol. Cell. Biol. 19: 989-1001) and by selecting the transformants capable of growing on a medium containing 1 mM of isoleucine (the strain EK011 which simultaneously carries a deletion in the GAP1 gene and a disruption of the AGP1 gene has very slow growth on a high concentration of isoleucine (1 mM). Moreover, in this strain the disruption of the AGP1 gene was carried out by the KanMX gene, the strain is therefore resistant to geneticin (Iraqui et al., 1999, Mol. Cell. Biol, 19, 989-1001). A transformant, CD225 which was capable of growing on 1 mM isoleucine and had in parallel become sensitive to geneticin was preserved. The replacement of the AGP1 gene by the proPGK-AGP1 gene was verified by PCR.

The CY131 strains were obtained by crossing the strains CC867-1D (Matα, his3, leu2, ura3, ade2, trp1, met32::TRP1, met30::URA3) and CY121-3D (Mata, his3, ura3, trp1, met32::TRP1, ado1::URA3, agp1::proPGK1-AGP1) and sporulating the diploids obtained, then by analyzing the tetrads obtained in order to select the spores having the desired gene recombinations.

The CY208 strains were obtained by crossing the strains CY188-9A (Mata, his3, trp1, ura3, met32::TRP1, ado1::URA3, met30::URA3, sam4::URA3, mht1::HIS3) and CY121-1C (Matα, his3, ura3, trp1, met32::TRP1, ado1::URA3, agp1::proPGK1-AGP1) and sporulating the diploids obtained, then by analyzing the tetrads obtained in order to select the spores having the desired gene recombinations.

The CY284 strains were obtained by crossing the strains CY273-2B (Mata, his3, leu2, ura3, trp1, met32::TRP1, met30::URA3, ado1::URA3, sam3-sam4::proPGK1-SAM2) and CY121-1C (Matα, his3, leu2, ura3, trp1, met32::TRP1, agp1::proPGK1-AGP1) and sporulating the diploids obtained, then by analyzing the tetrads obtained in order to select the spores having the desired gene recombinations.

### Example 6

### Analysis of the Effect of the ADE2 Mutation on the Production of SAM

Analysis of the production and excretion of SAM in the different strains which were constructed for this invention made it possible to observe that the production and excretion of SAM were systematically lower in strains carrying a mutation inactivating the ADE2 gene (a gene situated on the XV chromosome, coding for phosphoribosylaminoimidazole carboxylase) than in strains comprising an exactly equivalent genome but carrying a wild-type allele of the ADE2 gene. This mutation affects the biosynthesis of purines but its influence on the ability of the yeast cells to overproduce SAM remains difficult to explain. Nevertheless, in view of the results obtained, the strains constructed for the present invention will all carry a non-mutated wild-type allele of the ADE2 gene.

### Example 7

### Culture of the Genetically Modified Yeast Strains of the Invention in an Economically Advantageous Culture Medium

The cultures of the haploid strains of genotype CY208-7B and CY208-11B, CY273-9D and CY273-2B, CY284-9B and CY284-2B and of the diploid strains CY303-1 and CY303-4, and CY304-1 and CY304-2 (see Table 1), in an economically advantageous culture medium (containing between 3 and 10% ammonium phosphate, between 0.5 and 5% ammonium sulphate, between 0.1% and 1% magnesium sulphate, between 0.5 and 4% sodium citrate, between 5 and 10% glucose and between 0.1 and 2% DL-methionine) made it possible to obtain on average approximately 8 g of SAM per litre in 24 hours (see Table 1). Approximately 40% of this SAM produced is excreted in the culture medium and can therefore be purified without rupture of the cells.

The CY208 strains were obtained by crossing the strains (Mata, his3, trp1, ura3, met32::TRP1, ado1::URA3, met30::URA3, sam4::URA3, mht1::HIS3) and CY121-1C CY121-1C (Matα, his3, leu2, ura3, trp1, met32::TRP1, agp1::proPGK1-AGP1) and sporulating the diploids obtained, then by analyzing the tetrads obtained in order to select the spores having the desired gene recombinations.

The CY273 strains were obtained by crossing the strains CY270-7D (Mata, his3, leu2, ura3, trp1, met32::TRP1, ado1::URA3, sam3-sam4::proPGK1-SAM2) and CY258-10D (Matα, his3, leu2, ura3, trp1, met32::TRP1, met30::URA3, ado1::URA3, sam3-sam4::proSAM4-SAM2, mht1) and sporulating the diploids obtained, then by analyzing the tetrads obtained in order to select the spores having the desired gene recombinations.

The CY284 strains were obtained by crossing the strains CY273-2B (Mata, his3, leu2, ura3, trp1, met32::TRP1, met30::URA3, ado1::URA3, sam3-sam4::proPGK1-SAM2) and CY121-1C (Matα, his3, leu2, ura3, trp1, met32::TRP1, agp1::proPGK1-AGP1) and sporulating the diploids obtained, then by analyzing the tetrads obtained in order to select the spores having the desired gene recombinations.

TABLE 1
genotype of the strains obtained and associated SAM production and excretion profile

| | | Total SAM | Excreted SAM | Excretion |
|---|---|---|---|---|
| Strain | Genotype | (g/l/24 h) | (g/l/24 h) | % |
| CC788-2B | MATa, his3, leu2, ura3, ade2, trp1 | 0.229 | 0.016 | 7 |
| (reference strain) | | | | |
| CY78-3B | MATa, his3, leu2, ura3, ade2, trp1, | 2.85 | 0.60 | 21 |
| ado1Δ | ado1::URA3 | | | |
| CY168-1C | MATa, his3, leu2, ura3, trp1, sam4::URA3, | 2.41 | 1.13 | 47 |
| ado1Δ sam4Δ mht1Δ | mht1::HIS3, ado1::URA3 | | | |
| CY154-17A | MATa, his3, leu2, ura3, trp1, met32::TRP1, | 3.01 | 1.96 | 59 |
| met32Δ met30Δ ado1Δ | met30::URA3, ado1::URA3 | | | |
| CY131-14A | MATa, his3, ura3, trp1, met32::TRP1, | 4.88 | 1.95 | 40 |
| met32Δ met30Δ ado1Δ | met30::URA3, ado1::URA3, | | | |
| agp1::proPGK1-AGP1 | agp1::proPGK1-AGP1 | | | |
| CY188-9A | MATa, his3, leu2, ura3, trp1, sam4::URA3, | 6.32 | 3.69 | 58 |
| ado1Δ sam4Δ mht1Δ | mht1::HIS3, met32::TRP1, met30::URA3, | | | |
| met32A met30Δ | ado1::URA3 | | | |
| CY208-7B | MATa, his3, ura3, trp1, sam4::URA3, | 7.16 | 2.4 | 33 |
| ado1Δ sam4Δ mht1Δ | mht1::HIS3, met32::TRP1, met30::URA3, | | | |
| met32Δ met30Δ | ado1::URA3, agp1::proPGK1-AGP1 | | | |
| agp1::proPGK1-AGP1 | | | | |
| CY208-11D | MATa, his3, ura3, trp1, sam4::URA3, | 7.74 | 3.21 | 41 |
| ado1Δ sam4Δ mht1Δ | mht1::HIS3, met32::TRP1, met30::URA3, | | | |
| met32Δ met30Δ | ado1::URA3, agp1::proPGK1-AGP1 | | | |
| agp1::proPGK1-AGP1 | | | | |
| CY273-9D | MATa, his3, leu2, ura3, trp1, sam4::URA3, | 8.32 | 4.90 | 59 |
| ado1Δ sam4Δ mht1Δ | mht1::HIS3, met32::TRP1, met30::URA3, | | | |
| met32Δ met30Δ | ado1::URA3, sam3-sam4::proPGK-SAM2 | | | |
| sam3-sam4::proPGK-SAM2 | | | | |
| CY273-2B | MATa, his3, leu2, ura3, trp1, sam4::URA3, | 8.29 | 4.56 | 55 |
| ado1Δ sam4Δ mht1Δ | mht1::HIS3, met32::TRP1, met30::URA3, | | | |
| met32Δ met30Δ | ado1::URA3, sam3-sam4::proPGK-SAM2 | | | |
| sam3-sam4::proPGK-SAM2 | | | | |
| CY284-9B | MATa, his3, ura3, trp1, sam4::URA3, | 8.54 | 4.54 | 53 |
| ado1Δ sam4Δ mht1Δ | mht1::HIS3, met32::TRP1, met30::URA3, | | | |
| met32Δ met30Δ | ado1::URA3, sam3-sam4::proPGK-SAM2, | | | |
| sam3-sam4::proPGK-SAM2 | agp1::proPGK-AGP1. | | | |
| agp1::proPGK1-AGP1 | | | | |
| CY284-2B | MATa, his3, ura3, trp1, sam4::URA3, | 8.29 | 4.56 | 55 |
| ado1Δ sam4Δ mht1Δ | mht1::HIS3, met32::TRP1, met30::URA3, | | | |
| met32Δ met30Δ | ado1::URA3, | | | |
| sam3-sam4::proPGK-SAM2 | sam3-sam4::proPGK-SAM2, | | | |
| agp1::proPGK1-AGP1 | agp1::proPGK-AGP1. | | | |
| CY303-4 | MATa/MATa, his3/his3, leu2/leu2, | 6.12 | 3.88 | 63 |
| (CY273-9D × CY273-9D) | ura3/ura3, trp1/trp1, | | | |
| | sam4::URA3/sam4::URA3, | | | |
| | mht1::HIS3/mht1::HIS3, | | | |
| | met32::TRP1/met32::TRP1, | | | |
| | met30::URA3/met30::URA3, | | | |
| | ado1::URA3/ado1::URA3, sam3- | | | |
| | sam4::proPGK-SAM2/sam3- | | | |
| | sam4::proPGK-SAM2. | | | |
| CY304-2 | MATa/MATa, his3/his3, leu2/leu2, | 7.49 | 4.4 | 59 |
| (CY284-2B × CY284-9B) | ura3/ura3, trp1/trp1, | | | |
| | sam4::URA3/sam4::URA3, | | | |
| | mht1::HIS3/mht1::HIS3, | | | |
| | met32::TRP1/met32::TRP1, | | | |
| | met30::URA3/met30::URA3, | | | |
| | ado1::URA3/ado1::URA3, sam3- | | | |
| | sam4::proPGK-SAM2/sam3- | | | |
| | sam4::proPGK-SAM2, agp1::proPGK- | | | |
| | AGP1/agp1::proPGK-AGP1. | | | |

1-24. (canceled)

25. A Method for the production of S-adenosylmethionine (SAM) comprising culturing of a genetically modified yeast strain, in which the gene coding for adenosine kinase has been inactivated by genetic modification to product SAM.

26. The method according to claim 25, wherein the sequence of the gene coding for adenosine kinase of said strain has been disrupted.

27. The method according to claim 25, in which said strain has at least one other genetic modification chosen from the group comprising:
the inactivation of a gene chosen from the group comprising the gene coding for the high-affinity transporter of S-adenosylmethionine, the gene coding for S-adenosylmethionine-homocysteine methyl transferase, the gene coding for S-methylmethionine-homocysteine methyl transferase, and the gene coding for the Met30 receptor sub-unit of the ubiquitin ligase complex SCF^{Met30},
the introduction of an additional copy of the sequence of a gene chosen from the group comprising the gene coding for S-adenosylmethionine synthetase 1, the gene coding for S-adenosylmethionine synthetase 2, the gene coding for low-affinity methionine permease, the gene coding for high-affinity methionine permease, the gene coding for very low-affinity methionine permease, a gene coding for a broad-spectrum permease which can transport methionine, in the genome of said strain, and
the mutation of the promoter sequence of a gene chosen from the group comprising the gene coding for S-adenosylmethionine synthetase 1, the gene coding for S-adenosylmethionine synthetase 2, the gene coding for low-affinity methionine permease, the gene coding for high-affinity methionine permease, the gene coding for very low-affinity methionine permease, a gene coding for a broad-spectrum permease which can transport methionine.

28. The method according to claim 27, wherein the sequence of at least one of the genes chosen from the group comprising the gene coding for the high-affinity transporter of S-adenosylmethionine, the gene coding for S-adenosylmethionine-homocysteine methyl transferase, the gene coding for S-methylmethionine-homocysteine methyl transferase, and the gene coding for the Met30 receptor sub-unit of the ubiquitin ligase complex SCF^{Met30}, has been disrupted.

29. The method according to claim 27, wherein at least one promoter sequence of one of the genes of said strain chosen from the group comprising the gene coding for S-adenosylmethionine synthetase 1, the gene coding for S-adenosylmethionine synthetase 2, the gene coding for low-affinity methionine permease, the gene coding for high-affinity methionine permease, the gene coding for very low-affinity methionine permease, a gene coding for a broad-spectrum permease which can transport methionine, has been substituted by a strong promoter sequence of yeast.

30. The method according to claim 27, wherein the genes coding for the Met30 receptor sub-unit of the ubiquitin ligase complex SCF^{Met30}, S-methylmethionine-homocysteine methyl transferase, adenosine kinase, and S-adenosylmethionine-homocysteine methyl transferase of said strain are inactivated, (in particular) by disruption of the sequences of said genes.

31. The method according to claim 27, wherein an additional copy of the sequence of the gene coding for S-adenosylmethionine synthetase 2, coupled with a strong promoter, has been introduced into the genome of said strain.

32. The method according to claim 27, wherein the promoter sequence of the gene coding for low-affinity methionine permease of said strain has been substituted by the strong promoter sequence.

33. The method according to claim 27, wherein the gene coding for the high-affinity transporter of S-adenosylmethionine and the gene coding for S-adenosylmethionine-homocysteine methyl transferase of said strain have been inactivated by substitution of the sequence of said genes by a copy of the sequence of the gene coding for S-adenosylmethionine synthetase 2, coupled with a strong promoter.

34. The method according to 25, wherein the strong promoter is chosen from the group comprising the natural promoters of the PGK1, ADH1, TDH3, TEF1, PHO5, LEU2, and GAL1 genes of said strain.

35. The method according to claim 25, wherein said strain is prototrophic for adenine.

36. The method according to claim 25, wherein said strain is haploid.

37. The method according to claim 25, wherein said strain is diploid.

38. The method according to claim 25, wherein when the genetic modifications are chromosomic, said genetic modifications are carried by each of the two homologous chromosomes.

39. The method according to claim 25, wherein said strain does not comprise heterologous nucleotide sequences.

40. The method according to claim 25, wherein said genera Saccharomyces, Candida, Pichia, Schizosaccharomyces, and Kluyveromyces, and that said strain is in particular a yeast of the species Saccharomyces cerevisiae.

41. The method according to claim 27, wherein the strain belongs to the species Saccharomyces cerevisiae and wherein when the gene coding for the Met30 receptor sub-unit of the ubiquitin ligase complex SCF^{Met30} (MET30) of said strain is inactivated, by disruption of the sequence of the MET30 gene, then the MET4 gene and/or the MET32 gene of said strain is also inactivated, by disruption of the corresponding gene sequences.

42. Genetically modified yeast strain exhibiting increased production and excretion of S-adenosylmethionine compared with the corresponding non-modified yeast strain, said genetically modified strain according to claim 27.

43. A production process for S-adenosylmethionine, wherein it comprises the stages of:
culture of a genetically modified yeast strain according to claim 27 in a culture medium,
purification of S-adenosylmethionine from the supernatant of the culture medium and/or from the genetically modified yeast cells.

44. The process according to claim 43, the culture is carried out in a chemostat.

45. A pharmaceutical composition, wherein it comprises as active ingredient at least one yeast strain according to claim 25, in combination with a pharmaceutically acceptable vehicle.

46. A method for the treatment of diseases requiring an increased supply of S-adenosylmethionine, chosen from depression, arthritis, fibromyalgia, or male sterility comprising the administration of a yeast strain according to claim 25 to a patient in need thereof.

47. A method for the preparation of foods or drinks enriched with S-adenosylmethionine by means of a yeast strain according to claim 25.

48. Food preparation or drink, intended for human or animal consumption, comprising at least one genetically modified yeast strain according to claim 25.
